SEQUENCE LISTING (73 sequences)

SEQ ID NO: 1 - DNA, 15 bp, artificial
  initial oligonucleotide
tttccgcacgctacc

SEQ ID NO: 2 - DNA, 39 bp, artificial
  oligo GAPDH1
gctctctgctcctcctgttcgacaggtagcgtgcggaaa

SEQ ID NO: 3 - DNA, 41 bp, artificial
  oligo GAPDH2
gccatcaatgaccccttcattgacctggtagcgtgcggaaa

SEQ ID NO: 4 - DNA, 35 bp, artificial
  oligo GAPDH3
gctggcgctgagtacgtcgtggtagcgtgcggaaa

SEQ ID NO: 5 - DNA, 37 bp, artificial
  oligo GAPDH4
ccaactgcttagcacccctggcggtagcgtgcggaaa

SEQ ID NO: 6 - DNA, 36 bp, artificial
  oligo GAPDH5
ccaaggctgtgggcaaggtcaggtagcgtgcggaaa

SEQ ID NO: 7 - DNA, 38 bp, artificial
  oligo GAPDH6
cctcaagggcatcctgggctacaggtagcgtgcggaaa

SEQ ID NO: 8 - DNA, 35 bp, artificial
  oligo GAPDH7
gggtggtggacctcatggccggtagcgtgcggaaa

SEQ ID NO: 9 - DNA, 40 bp, artificial
  oligo GAPDH8
gagccgcaccttgtcatgtaccatcggtagcgtgcggaaa

SEQ ID NO: 10 - DNA, 37 bp, artificial
  oligo MALAT1
ggcgccgggaagcctcagctcgggtagcgtgcggaaa

SEQ ID NO: 11 - DNA, 48 bp, artificial
  oligo MALAT2
ggccacttgaactcgctttccatggcgatttgcggtagcgtgcggaaa

SEQ ID NO: 12 - DNA, 46 bp, artificial
  oligo MALAT3
gttgggggagaaagtccgccattttgccactggtagcgtgcggaaa

SEQ ID NO: 13 - DNA, 41 bp, artificial
  oligo MALAT4
gcctccctcacaaaggcggcggaaggggtagcgtgcggaaa

SEQ ID NO: 14 - DNA, 47 bp, artificial
  oligo MALAT5
ggctcctggagacacgacataaccaggagggtggtagcgtgcggaaa

SEQ ID NO: 15 - DNA, 37 bp, artificial
  oligo MALAT6
ggcagccagcgcaggggcttctggtagcgtgcggaaa

SEQ ID NO: 16 - DNA, 43 bp, artificial
  oligo MALAT7
ggactgaggagcaagcgagcaagcagcaggtagcgtgcggaaa

SEQ ID NO: 17 - DNA, 39 bp, artificial
  oligo MALAT8
ggtagcaggcggcttggcttggcaggtagcgtgcggaaa

SEQ ID NO: 18 - DNA, 48 bp, artificial
  oligo MALAT9
gcgagtggttggtaaaaatccgtgaggtcggcaggtagcgtgcggaaa

SEQ ID NO: 19 - DNA, 49 bp, artificial
  oligo MALAT10
gggatggtcttaacagggaagagagagggtggggggtagcgtgcggaaa

SEQ ID NO: 20 - DNA, 49 bp, artificial
  oligo MALAT11
ggcaattagttggcagtggcctgttacggttgggggtagcgtgcggaaa

SEQ ID NO: 21 - DNA, 42 bp, artificial
  oligo MALAT12
ggggttggtctggcctactgggctgacggtagcgtgcggaaa

SEQ ID NO: 22 - DNA, 46 bp, artificial
  oligo MALAT13
gagggtgggcttttgttgatgagggaggggaggtagcgtgcggaaa

SEQ ID NO: 23 - DNA, 48 bp, artificial
  oligo MALAT14
gggatcaagtggattgaggaggctgtgctgtgtggtagcgtgcggaaa

SEQ ID NO: 24 - DNA, 50 bp, artificial
  oligo MALAT15
cctgaccccttccctaggggatttcaggattgagaggtagcgtgcggaaa

SEQ ID NO: 25 - DNA, 39 bp, artificial
  oligo MALAT16
gggaagggagggggtgcctgtgggggtagcgtgcggaaa

SEQ ID NO: 26 - DNA, 48 bp, artificial
  oligo MALAT17
tctgtagttcagtgttggggcaatcttggggggggtagcgtgcggaaa

SEQ ID NO: 27 - DNA, 45 bp, artificial
  oligo MALAT18
tcctggaatttggagggatgggaggaggggggtagcgtgcggaaa

SEQ ID NO: 28 - DNA, 47 bp, artificial
  oligo MALAT19
gcagacacacgtatgcgaagggccagagaagcggtagcgtgcggaaa

SEQ ID NO: 29 - DNA, 40 bp, artificial
  oligo MALAT20
ggaggggtgaggtgggcgctaagccggtagcgtgcggaaa

SEQ ID NO: 30 - DNA, 42 bp, artificial
  oligo MALAT21
gcggtgcttgaaggggagggaaaggggggtagcgtgcggaaa

SEQ ID NO: 31 - DNA, 47 bp, artificial
  oligo MALAT22
gagtggctgagagggcttttgggtgggaatgcggtagcgtgcggaaa

SEQ ID NO: 32 - DNA, 48 bp, artificial
  oligo MALAT23
tggagttttggggaggtgggaggtaacagcacaggtagcgtgcggaaa

SEQ ID NO: 33 - RNA, 1285 nt, homo sapiens
gcucucugcuccuccuguucgacagucagccgcaucuucuuuugcgucgccagccgagccacaucgcucagacaccauggggaaggugaaggucggagucaacggauuuggucguauugggcgccuggucaccagggcugcuuuuaacucugguaaaguggauauuguugccaucaaugaccccuucauugaccucaacuacaugguuuacauguuccaauaugauuccacccauggcaaauuccauggcaccgucaaggcugagaacgggaagcuugucaucaauggaaaucccaucaccaucuuccaggagcgagaucccuccaaaaucaaguggggcgaugcuggcgcugaguacgucguggaguccacuggcgucuucaccaccauggagaaggcuggggcucauuugcaggggggagccaaaagggucaucaucucugcccccucugcugaugcccccauguucgucaugggugugaaccaugagaaguaugacaacagccucaagaucaucagcaaugccuccugcaccaccaacugcuuagcaccccuggccaaggucauccaugacaacuuugguaucguggaaggacucaugaccacaguccaugccaucacugccacccagaagacuguggauggccccuccgggaaacuguggcgugauggccgcggggcucuccagaacaucaucccugccucuacuggcgcugccaaggcugugggcaaggucaucccugagcugaacgggaagcucacuggcauggccuuccguguccccacugccaacgugucagugguggaccugaccugccgucuagaaaaaccugccaaauaugaugacaucaagaagguggugaagcaggcgucggagggcccccucaagggcauccugggcuacacugagcaccagguggucuccucugacuucaacagcgacacccacuccuccaccuuugacgcuggggcuggcauugcccucaacgaccacuuugucaagcucauuuccugguaugacaacgaauuuggcuacagcaacagggugguggaccucauggcccacauggccuccaaggaguaagaccccuggaccaccagccccagcaagagcacaagaggaagagagagacccucacugcuggggagucccugccacacucagucccccaccacacugaaucuccccuccucacaguugccauguagaccccuugaagaggggaggggccuagggagccgcaccuugucauguaccaucaauaaaguacccugugcucaacca

SEQ ID NO: 34 - RNA, 8779 nt, homo sapiens
cgcagccugcagcccgagacuucuguaaaggacuggggccccgcaacuggccucuccugcccucuuaagcgcagcgccauuuuagcaacgcagaagcccggcgccgggaagccucagcucgccugaaggcagguccccucugacgccuccgggagcccagguuucccagaguccuugggacgcagcgacgaguugugcugcuaucuuagcuguccuuauaggcuggccauuccagguggugguauuuagauaaaaccacucaaacucugcaguuuggucuugggguuuggaggaaagcuuuuauuuuucuuccugcuccgguucagaaggucugaagcucauaccuaaccaggcauaacacagaaucugcaaaacaaaaaccccuaaaaaagcagacccagagcaguguaaacacuucugggugugucccugacuggcugcccaaggucucugugucuucggagacaaagccauucgcuuaguuggucuacuuuaaaaggccacuugaacucgcuuuccauggcgauuugccuugugagcacuuucaggagagccuggaagcugaaaaacgguagaaaaauuuccgugcgggccguggggggcuggcggcaacuggggggccgcagaucagagugggccacuggcagccaacggcccccggggcucaggcggggagcagcucuguggugugggauugaggcguuuuccaagaguggguuuucacguuucuaagauuucccaagcagacagcccgugcugcuccgauuucucgaacaaaaaagcaaaacguguggcugucuugggagcaagucgcaggacugcaagcaguugggggagaaaguccgccauuuugccacuucucaaccgucccugcaaggcuggggcucaguugcguaauggaaaguaaagcccugaacuaucacacuuuaaucuuccuucaaaaggugguaaacuauaccuacugucccucaagagaacacaagaagugcuuuaagagguauuuuaaaaguuccggggguuuugugagguguuugaugacccguuuaaaauaugauuuccauguuucuuuugucuaaaguuugcagcucaaaucuuuccacacgcuaguaauuuaaguauuucugcauguguaguuugcauucaaguuccauaagcuguuaagaaaaaucuagaaaaguaaaacuagaaccuauuuuuaaccgaagaacuacuuuuugccucccucacaaaggcggcggaaggugaucgaauuccggugaugcgaguuguucuccgucuauaaauacgccucgcccgagcugugcgguaggcauugaggcagccagcgcaggggcuucugcugagggggcaggcggagcuugaggaaaccgcagauaaguuuuuuucucuuugaaagauagagauuaauacaacuacuuaaaaaauauagucaauagguuacuaagauauugcuuagcguuaaguuuuuaacguaauuuuaauagcuuaagauuuuaagagaaaauaugaagacuuagaagaguagcaugaggaaggaaaagauaaaagguuucuaaaacaugacggagguugagaugaagcuucuucauggaguaaaaaauguauuuaaaagaaaauugagagaaaggacuacagagccccgaauuaauaccaauagaagggcaaugcuuuuagauuaaaaugaaggugacuuaaacagcuuaaaguuuaguuuaaaaguuguaggugauuaaaauaauuugaaggcgaucuuuuaaaaagagauuaaaccgaaggugauuaaaagaccuugaaauccaugacgcagggagaauugcgucauuuaaagccuaguuaacgcauuuacuaaacgcagacgaaaauggaaagauuaauugggagugguaggaugaaacaauuuggagaagauagaaguuugaaguggaaaacuggaagacagaaguacgggaaggcgaagaaaagaauagagaagauagggaaauuagaagauaaaaacauacuuuuagaagaaaaaagauaaauuuaaaccugaaaaguaggaagcagaagaaaaaagacaagcuaggaaacaaaaagcuaagggcaaaauguacaaacuuagaagaaaauuggaagauagaaacaagauagaaaaugaaaauauugucaagaguuucagauagaaaaugaaaaacaagcuaagacaaguauuggagaaguauagaagauagaaaaauauaaagccaaaaauuggauaaaauagcacugaaaaaaugaggaaauuauugguaaccaauuuauuuuaaaagcccaucaauuuaauuucugguggugcagaaguuagaagguaaagcuugagaagaugaggguguuuacguagaccagaaccaauuuagaagaauacuugaagcuagaaggggaaguugguuaaaaaucacaucaaaaagcuacuaaaaggacugguguaauuuaaaaaaaacuaaggcagaaggcuuuuggaagaguuagaagaauuuggaaggccuuaaauauaguagcuuaguuugaaaaaugugaaggacuuucguaacggaaguaauucaagaucaagaguaauuaccaacuuaauguuuuugcauuggacuuugaguuaagauuauuuuuuaaauccugaggacuagcauuaauugacagcugacccaggugcuacacagaaguggauucagugaaucuaggaagacagcagcagacaggauuccaggaaccaguguuugaugaagcuaggacugaggagcaagcgagcaagcagcaguucguggugaagauaggaaaagaguccaggagccagugcgauuuggugaaggaagcuaggaagaaggaaggagcgcuaacgauuugguggugaagcuaggaaaaaggauuccaggaaggagcgagugcaauuuggugaugaagguagcaggcggcuuggcuuggcaaccacacggaggaggcgagcaggcguugugcguagaggauccuagaccagcaugccagugugccaaggccacagggaaagcgagugguugguaaaaauccgugaggucggcaauauguuguuuuucuggaacuuacuuaugguaaccuuuuauuuauuuucuaauauaaugggggaguuucguacugagguguaaagggauuuauauggggacguaggccgauuuccggguguuguagguuucucuuuuucaggcuuauacucaugaaucuugucugaagcuuuugagggcagacugccaaguccuggagaaauaguagauggcaaguuuguggguuuuuuuuuuuuacacgaauuugaggaaaaccaaaugaauuugauagccaaauugagacaauuucagcaaaucuguaagcaguuuguauguuuaguugggguaaugaaguauuucaguuuugugaauagaugaccuguuuuuacuuccucacccugaauucguuuuguaaauguagaguuuggauguguaacugaggcgggggggaguuuucaguauuuuuuuuugugggggugggggcaaaauauguuuucaguucuuuuucccuuaggucugucuagaauccuaaaggcaaaugacucaagguguaacagaaaacaagaaaauccaauaucaggauaaucagaccaccacagguuuacaguuuauagaaacuagagcaguucucacguugaggucuguggaagagauguccauuggagaaauggcugguaguuacucuuuuuuccccccacccccuuaaucagacuuuaaaagugcuuaaccccuuaaacuuguuauuuuuuacuugaagcauuuugggauggucuuaacagggaagagagagggugggggagaaaauguuuuuuucuaagauuuuccacagaugcuauaguacuauugacaaacuggguuagagaaggaguguaccgcugugcuguuggcacgaacaccuucagggacuggagcugcuuuuauccuuggaagaguauucccaguugaagcugaaaaguacagcacagugcagcuuugguucauauucagucaucucaggagaacuucagaagagcuugaguaggccaaauguugaaguuaaguuuuccaauaaugugacuucuuaaaaguuuuauuaaaggggaggggcaaauauuggcaauuaguuggcaguggccuguuacgguugggauuggugggguggguuuagguaauuguuuaguuuaugauugcagauaaacucaugccagagaacuuaaagucuuagaauggaaaaaguaaagaaauaucaacuuccaaguuggcaaguaacucccaaugauuuaguuuuuuuccccccaguuugaauugggaagcugggggaaguuaaauaugagccacuggguguaccagugcauuaauuugggcaaggaaagugucauaauuugauacuguaucuguuuuccuucaaaguauagagcuuuuggggaaggaaaguauugaacuggggguuggucuggccuacugggcugacauuaacuacaauuaugggaaaugcaaaaguuguuuggauaugguagugugugguucucuuuuggaauuuuuuucaggugauuuaauaauaauuuaaaacuacuauagaaacugcagagcaaaggaaguggcuuaaugauccugaagggauuucuucugaugguagcuuuuguauuaucaaguaagauucuauuuucaguuguguguaagcaaguuuuuuuuuaguguaggagaaauacuuuuccauuguuuaacugcaaaacaagauguuaagguaugcuucaaaaauuuuguaaauuguuuauuuuaaacuuaucuguuuguaaauuguaacugauuaagaauugugauaguucagcuugaaugucucuuagagggugggcuuuuguugaugagggaggggaaacuuuuuuuuuuucuauagacuuuuuucagauaacaucuucugagucauaaccagccuggcaguaugauggccuagaugcagagaaaacagcuccuuggugaauugauaaguaaaggcagaaaagauuauaugucauaccuccauuggggaauaagcauaacccugagauucuuacuacugaugagaacauuaucugcauaugccaaaaaauuuuaagcaaaugaaagcuaccaauuuaaaguuacggaaucuaccauuuuaaaguuaauugcuugucaagcuauaaccacaaaaauaaugaauugaugagaaauacaaugaagaggcaauguccaucucaaaauacugcuuuuacaaaagcagaauaaaagcgaaaagaaaugaaaauguuacacuacauuaauccuggaauaaaagaagccgaaauaaaugagagaugaguugggaucaaguggauugaggaggcugugcugugugccaauguuucguuugccucagacagguaucucuucguuaucagaagaguugcuucauuucaucugggagcagaaaacagcaggcagcuguuaacagauaaguuuaacuugcaucugcaguauugcauguuagggauaagugcuuauuuuuaagagcuguggaguucuuaaauaucaaccauggcacuuucuccugaccccuucccuaggggauuucaggauugagaaauuuuuccaucgagccuuuuuaaaauuguaggacuuguuccugugggcuucagugaugggauaguacacuucacucagaggcauuugcaucuuuaaauaauuucuuaaaagccucuaaagugaucagugccuugaugccaacuaaggaaauuuguuuagcauugaaucucugaaggcucuaugaaaggaauagcaugaugugcuguuagaaucagauguuacugcuaaaauuuacauguugugauguaaauuguguagaaaaccauuaaaucauucaaaauaauaaacuauuuuuauuagagaauguauacuuuuagaaagcugucuccuuauuuaaauaaaauaguguuugucuguaguucaguguuggggcaaucuugggggggauucuucucuaaucuuucagaaacuuugucugcgaacacucuuuaauggaccagaucaggauuugagcggaagaacgaauguaacuuuaaggcaggaaagacaaauuuuauucuucauaaagugaugagcauauaauaauuccaggcacauggcaauagaggcccucuaaauaaggaauaaauaaccucuuagacaggugggagauuaugaucagaguaaaagguaauuacacauuuuauuuccagaaagucaggggucuauaaauugacagugauuagaguaauacuuuuucacauuuccaaaguuugcauguuaacuuuaaaugcuuacaaucuuagagugguaggcaauguuuuacacuauugaccuuauauagggaagggagggggugccugugggguuuuaaagaauuuuccuuugcagaggcauuucauccuucaugaagccauucaggauuuugaauugcauaugagugcuuggcucuuccuucuguucuagugaguguaugagaccuugcagugaguuuaucagcauacucaaaauuuuuuuccuggaauuuggagggaugggaggaggggguggggcuuacuuguuguagcuuuuuuuuuuuuuacagacuucacagagaaugcaguugucuugacuucaggucugucuguucuguuggcaaguaaaugcaguacuguucugaucccgcugcuauuagaaugcauugugaaacgacuggaguaugauuaaaaguuguguuccccaaugcuuggaguagugauuguugaaggaaaaaauccagcugagugauaaaggcugaguguugaggaaauuucugcaguuuuaagcagucguauuugugauugaagcugaguacauuuugcugguguauuuuuagguaaaaugcuuuuuguucauuucugguggugggaggggacugaagccuuuagucuuuuccagaugcaaccuuaaaaucagugacaagaaacauuccaaacaagcaacagucuucaagaaauuaaacuggcaaguggaaauguuuaaacaguucagugaucuuuagugcauuguuuauguguggguuucucucuccccucccuuggucuuaauucuuacaugcaggaacacucagcagacacacguaugcgaagggccagagaagccagacccaguaagaaaaaauagccuauuuacuuuaaauaaaccaaacauuccauuuuaaauguggggauugggaaccacuaguucuuucagaugguauucuucagacuauagaaggagcuuccaguugaauucaccaguggacaaaaugaggaaaacaggugaacaagcuuuuucuguauuuacauacaaagucagaucaguuaugggacaauaguauugaauagauuucagcuuuaugcuggaguaacuggcaugugagcaaacuguguuggcguggggguggaggggugaggugggcgcuaagccuuuuuuuaagauuuuucagguaccccucacuaaaggcaccgaaggcuuaaaguaggacaaccauggagccuuccuguggcaggagagacaacaaagcgcuauuauccuaaggucaagagaagugucagccucaccugauuuuuauuaguaaugaggacuugccucaacucccucuuucuggagugaagcauccgaaggaaugcuugaaguaccccugggcuucucuuaacauuuaagcaagcuguuuuuauagcagcucuuaauaauaaagcccaaaucucaagcggugcuugaaggggagggaaagggggaaagcgggcaaccacuuuucccuagcuuuuccagaagccuguuaaaagcaaggucuccccacaagcaacuucucugccacaucgccaccccgugccuuuugaucuagcacagacccuucaccccucaccucgaugcagccaguagcuuggauccuugugggcaugauccauaaucgguuucaagguaacgauggugucgaggucuuugguggguugaacuauguuagaaaaggccauuaauuugccugcaaauuguuaacagaaggguauuaaaaccacagcuaaguagcucuauuauaauacuuauccagugacuaaaaccaacuuaaaccaguaaguggagaaauaacauguucaagaacuguaaugcugggugggaacauguaacuuguagacuggagaagauaggcauuugaguggcugagagggcuuuugggugggaaugcaaaaauucucugcuaagacuuuuucaggugaacauaacagacuuggccaagcuagcaucuuagcggaagcugaucuccaaugcucuucaguagggucaugaagguuuuucuuuuccugagaaaacaacacguauuguuuucucagguuuugcuuuuuggccuuuuucuagcuuaaaaaaaaaaaaagcaaaagaugcuggugguuggcacuccugguuuccaggacgggguucaaaucccugcggcgucuuugcuuugacuacuaaucugucuucaggacucuuucuguauuucuccuuuucucugcaggugcuaguucuuggaguuuuggggaggugggagguaacagcacaauaucuuugaacuauauacauccuugauguauaauuugucaggagcuugacuugauuguauauucauauuuacacgagaaccuaauauaacugccuugucuuuuucagguaauagccugcagcugguguuuugagaagcccuacugcugaaaacuuaacaauuuuguguaauaaaaauggagaagcucuaaauuguugugguucuuuugugaauaaaaaaaucuugauuggggaaaaaa

SEQ ID NO: 35 - DNA, 39 bp, artificial
  oligo GAPDH1p
tttccgcacgctacctgtcgaacaggaggagcagagagc

SEQ ID NO: 36 - DNA, 41 bp, artificial
  oligo GAPDH2p
tttccgcacgctaccaggtcaatgaaggggtcattgatggc

SEQ ID NO: 37 - DNA, 35 bp, artificial
  oligo GAPDH3p
tttccgcacgctaccacgacgtactcagcgccagc

SEQ ID NO: 38 - DNA, 37 bp, artificial
  oligo GAPDH4p
tttccgcacgctaccgccaggggtgctaagcagttgg

SEQ ID NO: 39 - DNA, 36 bp, artificial
  oligo GAPDH5p
tttccgcacgctacctgaccttgcccacagccttgg

SEQ ID NO: 40 - DNA, 38 bp, artificial
  oligo GAPDH6p
tttccgcacgctacctgtagcccaggatgcccttgagg

SEQ ID NO: 41 - DNA, 35 bp, artificial
  oligo GAPDH7b
tttccgcacgctaccggccatgaggtccaccaccc

SEQ ID NO: 42 - DNA, 40 bp, artificial
  oligo GAPDH8p
tttccgcacgctaccgatggtacatgacaaggtgcggctc

SEQ ID NO: 43 - DNA, 37 bp, artificial
  oligo MALAT1p
tttccgcacgctacccgagctgaggcttcccggcgcc

SEQ ID NO: 44 - DNA, 48 bp, artificial
  oligo MALAT2p
tttccgcacgctaccgcaaatcgccatggaaagcgagttcaagtggcc

SEQ ID NO: 45 - DNA, 46 bp, artificial
  oligo MALAT3p
tttccgcacgctaccagtggcaaaatggcggactttctcccccaac

SEQ ID NO: 46 - DNA, 41 bp, artificial
  oligo MALAT4p
tttccgcacgctaccccttccgccgcctttgtgagggaggc

SEQ ID NO: 47 - DNA, 47 bp, artificial
  oligo MALAT5p
tttccgcacgctaccaccctcctggttatgtcgtgtctccaggagcc

SEQ ID NO: 48 - DNA, 37 bp, artificial
  oligo MALAT6p
tttccgcacgctaccagaagcccctgcgctggctgcc

SEQ ID NO: 49 - DNA, 43 bp, artificial
  oligo MALAT7b
tttccgcacgctacctgctgcttgctcgcttgctcctcagtcc

SEQ ID NO: 50 - DNA, 39 bp, artificial
  oligo MALAT8b
tttccgcacgctacctgccaagccaagccgcctgctacc

SEQ ID NO: 51 - DNA, 48 bp, artificial
  oligo MALAT9p
tttccgcacgctacctgccgacctcacggatttttaccaaccactcgc

SEQ ID NO: 52 - DNA, 49 bp, artificial
  oligo MALAT10p
tttccgcacgctaccccccaccctctctcttccctgttaagaccatccc

SEQ ID NO: 53 - DNA, 49 bp, artificial
  oligo MALAT11p
tttccgcacgctacccccaaccgtaacaggccactgccaactaattgcc

SEQ ID NO: 54 - DNA, 42 bp, artificial
  oligo MALAT12p
tttccgcacgctaccgtcagcccagtaggccagaccaacccc

SEQ ID NO: 55 - DNA, 46 bp, artificial
  oligo MALAT13p
tttccgcacgctacctcccctccctcatcaacaaaagcccaccctc

SEQ ID NO: 56 - DNA, 48 bp, artificial
  oligo MALAT14p
tttccgcacgctaccacacagcacagcctcctcaatccacttgatccc

SEQ ID NO: 57 - DNA, 50 bp, artificial
  oligo MALAT15p
tttccgcacgctacctctcaatcctgaaatcccctagggaaggggtcagg

SEQ ID NO: 58 - DNA, 39 bp, artificial
  oligo MALAT16p
tttccgcacgctacccccacaggcaccccctcccttccc

SEQ ID NO: 59 - DNA, 48 bp, artificial
  oligo MALAT17p
tttccgcacgctaccccccccaagattgccccaacactgaactacaga

SEQ ID NO: 60 - DNA, 45 bp, artificial
  oligo MALAT18p
tttccgcacgctacccccctcctcccatccctccaaattccagga

SEQ ID NO: 61 - DNA, 47 bp, artificial
  oligo MALAT19p
tttccgcacgctaccgcttctctggcccttcgcatacgtgtgtctgc

SEQ ID NO: 62 - DNA, 40 bp, artificial
  oligo MALAT20p
tttccgcacgctaccggcttagcgcccacctcacccctcc

SEQ ID NO: 63 - DNA, 42 bp, artificial
  oligo MALAT21p
tttccgcacgctacccccctttccctccccttcaagcaccgc

SEQ ID NO: 64 - DNA, 47 bp, artificial
  oligo MALAT22p
tttccgcacgctaccgcattcccacccaaaagccctctcagccactc

SEQ ID NO: 65 - DNA, 48 bp, artificial
  oligo MALAT23p
tttccgcacgctacctgtgctgttacctcccacctccccaaaactcca

SEQ ID NO: 66 - DNA, 22 bp, artificial
  primer GAPDHqF
gtctcctctgacttcaacagcg

SEQ ID NO: 67 - DNA, 22 bp, artificial
  primer GAPDHqR
accaccctgttgctgtagccaa

SEQ ID NO: 68 - DNA, 20 bp, artificial
  primer MALAT1qF
gacggaggttgagatgaagc

SEQ ID NO: 69 - DNA, 20 bp, artificial
  primer MALAT1qR
attcggggctctgtagtcct

SEQ ID NO: 70 - DNA, 20 bp, artificial
  primer hm18SqF
gtaacccgttgaaccccatt

SEQ ID NO: 71 - DNA, 20 bp, artificial
  primer hm18SqR
ccatccaatcggtagtagcg

SEQ ID NO: 72 - DNA, 17 bp, artificial
  primer hACTqF
aggcaccagggcgtgat

SEQ ID NO: 73 - DNA, 23 bp, artificial
  primer hACTqR
gcccacataggaatccttctgac

(19) United States
(12) Patent Application Publication
(10) Pub. No.: US 2023/0159993 A1
(43) Pub. Date: May 25, 2023

(54) Particles for Capture of Nucleic Acid Molecules

(71) Applicant: ELEMENTZERO BIOLABS UG, Berlin (DE)

(72) Inventor: Karol Jerzy Rogowski, Berlin (DE)

(21) Appl. No.: 17/920,983

(22) PCT Filed: May 25, 2021
(86) PCT No.: PCT/EP2021/063800

§ 371 Date: Oct. 24, 2022

(30) Foreign Application Priority Data
May 25, 2020 (EP) 20176351.3

(51) Int. Cl. C12Q 1/6834

(52) U.S. Cl. CPC C12Q 1/6834; C12Q 1/6851

(57) ABSTRACT

The present invention relates to the field of nucleic acid capture. The present invention inter alia concerns methods of producing a surface on which multiple copies of each of multiple DNA oligonucleotide species are covalently attached. Particles on the surface of which multiple copies of each of multiple DNA oligonucleotide species are covalently attached are also disclosed. Further, the present invention provides methods for enriching or depleting one or more species of nucleic acid molecules in/from a sample, including in/from partially isolated nucleic acids, isolated nucleic acids, biological samples, crude tissue lysates, cleared tissue lysates, crude cell lysates, cleared cell lysates, and processed and amplified nucleic acid sequencing libraries.

### FIELD OF THE INVENTION

The present invention relates to the field of nucleic acid capture. The present invention inter alia concerns methods of producing a surface on which multiple copies of each of multiple DNA oligonucleotide species are covalently attached. Particles on the surface of which multiple copies of each of multiple DNA oligonucleotide species are covalently attached are also disclosed. Further, the present invention provides methods for enriching or depleting one or more species of nucleic acid molecules in/from a sample, including in/from partially isolated nucleic acids, isolated nucleic acids, biological samples, crude tissue lysates, cleared tissue lysates, crude cell lysates, cleared cell lysates, and processed and amplified nucleic acid sequencing libraries.

### BACKGROUND OF THE INVENTION

The concept of using solid surfaces, including paramagnetic particles, coupled to different molecules in order to capture nucleic acids is known in the art. However, severe limitations exist when capture of specific RNA or DNA molecule is considered.

Nucleic acids can be non-discriminately captured using paramagnetic particles that utilize the fact that nucleic acids bear an electric charge, which, under specific conditions, lets them reversibly bind to the surface of these particles. This approach provides a way to capture nucleic acids with a specific length cutoff, which cutoff can be provided by modifications of the environment under which particles interact with nucleic acids. It does not provide, however, any additional restrictions to the nature or specificity of the capture.

A paramagnetic particle-based solution, which allows the capture of nucleic acids of a single defined oligonucleotide sequence has also been developed in the past. In this approach, an oligonucleotide sequence is synthesized directly on the appropriately modified surface of the particle. The resulting particles bear the oligonucleotide sequence cross-linked permanently to their surface. These particles can be conveniently employed in a rapid hybridization based capture of nucleic acids comprising a sequence that is complementary to the oligonucleotide sequence that the particles carry. However, due to the nature of the chemical oligonucleotide synthesis, only a single defined oligonucleotide sequence can be synthesized on the surface of paramagnetic particles (synthesis of different sequences in a single synthesis reaction is possible on solid, immobile surfaces, but not on surfaces that are mobile in a solution). That is, the generated particles will carry oligonucleotides that all have the same nucleotide sequence.

The main limitation in usefulness of such particles lays in the nature of a typical sample in which nucleic acids are to be captured. In such a sample, the efficiency of using a single defined oligonucleotide sequence to capture a nucleic acid molecule is directly related to the abundance of the nucleic acid to be purified, i.e. of the sequence that is complementary to the sequence of the oligonucleotide cross-linked to the particle, in the sample. Typically, sequences that are not abundant in the sample cannot be captured reliably. Therefore, such particles are widely used only for purification of mRNAs by the capture of their poly A tail, where the capture particles carry an oligo dT sequence, and is not commonly employed for capturing other nucleic acid sequences.

A group of biotin based approaches utilizing paramagnetic particles have also been developed. While varied, they are all based on the use of free oligonucleotides labeled with a biotin molecule on their 3′ or 5′ end. These labeled oligonucleotides (probes) are hybridized with complementary sequences (targets) in the nucleic acid sample in which nucleic acids are to be captured. After hybridization, the target-probe complexes can bind to avid in via the biotin label present on one of the ends of the probe. Typically, paramagnetic particles coupled to avid in molecules are used to capture the target nucleic acid. Methodologies based on this concept are commonly used, but have significant drawbacks:

1) Binding of the target to the particles is a two-step procedure, i.e. first hybridization of target to probe has to occur so that the hybridized complex can then be captured by the particles. This leads to a more laborious capture procedure, increases the potential experimental bias, and requires careful consideration of the ratio of biotin to avidin molecules present in the experimental design.
2) The capture can only take place under conditions that are favorable for the binding of biotin to avid in.
3) Naturally occurring biotin molecules may be present in the sample in which nucleic acid molecules are to be captured, interfering with the target capture via the avidin-coupled particles.
4) The cost of biotinylated oligonucleotides is relatively high.

Most relevant examples of biotinylated oligonucleotides based RNA capture methodologies include: Capture Hybridization Analysis of RNA Targets (CHART), RNA Antisense Purification (RAP) and Chromatin Isolation by RNA Purification (ChIRP) (as described in Simon et al., 2011, Proc Natl Acad Sci USA 108, 20497-20502; Engreitz et al., 2013; Science 341, 1237973; Chu et al., 2011, Mol. Cell 44, 667-678).

Accordingly, there is a need for improved methods of capturing nucleic acid molecules in varied samples under varied conditions that are straight-forward to perform and reasonably priced while providing specific and efficient capture of specific nucleic acid species.

### SUMMARY OF THE INVENTION

The present invention solves the above need by inter alia providing methods and particles for enriching or depleting one or more species of nucleic acid molecules from a sample, as well as by providing methods for producing such particles. The inventive approach allows, for the first time, for the use of a set of hybridization probes comprising different sequences covalently attached to particles for the capture of target nucleic acid molecules in a cost efficient way. This approach has significant advantages over previously used methodologies:

1) It allows for simultaneous use of multiple different capture probes of different nucleotide sequences.
2) The capture is a one-step procedure (probes are covalently pre-coupled to the particles, so that hybridization of probe to target and capture of the hybridized target occur simultaneously. The capture therefore occurs quickly, with limited hands-on time required, and the capture efficiency is only limited by the probe-to target base pairing efficiency and not by additional molecular interactions.
3) It allows for the use of very strong chemical denaturing and reducing conditions in the sample in which nucleic acids are to be captured, since only the oligonucleotide base pairing properties have to be maintained throughout the capture.
4) It alleviates the need for costly chemical modifications of the probe oligonucleotides.
5) Due to the fact that capture probes with different sequences targeting the same nucleic acid molecule are grouped in close proximity on the surface of each particle, they are capable of working in synergy. Binding of a complementary sequence within the target nucleic acid by a single capture probe automatically brings the target nucleic acid into close proximity with other capture probes of different oligonucleotide species, i.e. with other sequences targeting the same target nucleic acid molecule at different sequence stretches that are complimentary to the sequences of these other capture probes. Thus, the different capture probe species reinforce each other's binding efficiency.

In the following, the aspects of the invention are described. Embodiments of these aspects are also mentioned.

### First Aspect: Method for Producing a Surface on which Multiple Copies of Each of Multiple DNA Oligonucleotide Species are Covalently Attached

In a first aspect, the present invention provides a method of producing a surface on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a surface on which multiple copies of an initial DNA oligonucleotide are covalently attached at their 5′ ends, wherein the initial oligonucleotide has a predetermined nucleotide sequence;
   ii. a DNA-dependent DNA polymerase;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the surface, and
      a 5′ sequence that is unique for each of the multiple free oligonucleotide species;
b. hybridizing the multiple copies of the multiple free oligonucleotide species to the multiple copies of the initial oligonucleotide that is covalently attached to the surface at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the surface at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and
e. separating the surface from the denatured reaction mixture of step d.

In an embodiment, the surface is the surface of a particle. In one such embodiment, the particle is a magnetic particle. In one such embodiment, the magnetic particle is a paramagnetic particle, and the separating in step e is magnetically separating.

In an embodiment, the DNA-dependent DNA polymerase is selected from the group consisting of DNA-dependent DNA polymerase that produces blunt ends and DNA-dependent DNA polymerase that produce sticky ends. In one such embodiment, the DNA-dependent DNA polymerase is a DNA-dependent DNA polymerase that produces blunt ends.

In an embodiment,
the first temperature is from 25° C. to 72° C. and/or
the second temperature is from 40° C. to 78° C., optionally from 60° C. to 78° C., and/or
the third temperature is from 90° C. to 98° C.

In one such embodiment, both the first and second temperatures are from 40° C. to 72° C. and steps b and c are performed concurrently.

In an embodiment, the initial oligonucleotide that is covalently attached to the surface is from 5 nucleotides to 100 nucleotides in length. In one such embodiment, the initial oligonucleotide that is covalently attached to the surface is from 10 nucleotides to 20 nucleotides in length.

In an embodiment, the free oligonucleotide species are from 10 nucleotides to 1000 nucleotides in length. In one such embodiment, the free oligonucleotide species are from 24 nucleotides to 50 nucleotides, in length.

In an embodiment, the 3′ sequence of the free oligonucleotide species that is complimentary to the 3′ sequence of the covalently attached initial oligonucleotide is from 5 nucleotides to 100 nucleotides in length. In one such embodiment, the 3′ sequence of the free oligonucleotide species that is complimentary to the 3′ sequence of the covalently attached initial oligonucleotide is from 10 nucleotides to 20 nucleotides, in length.

### Second Aspect: Particle on the Surface of which Multiple Copies of Each of Multiple DNA Oligonucleotide Species are Covalently Attached

In a second aspect, the present invention provides a particle on the surface of which multiple copies of each of multiple DNA oligonucleotide species are covalently attached at their 5′ ends, wherein the oligonucleotide species each have a predetermined nucleotide sequence, and wherein the predetermined nucleotide sequence of each oligonucleotide species comprises a 3′ sequence that is unique for each of the oligonucleotide species.

In an embodiment, the particle is a magnetic particle. In one such embodiment, the magnetic particle is a paramagnetic particle.

In an embodiment, the DNA oligonucleotide species are from 10 nucleotides to 1000 nucleotides in length. In one such embodiment, the DNA oligonucleotide species are from 24 nucleotides to 70 nucleotides, in length.

In an embodiment, the unique 3′ sequence is from 5 nucleotides to 995 nucleotides in length. In one such embodiment, the unique 3′ sequence is from 12 nucleotides to 50 nucleotides, in length.

### Third Aspect: Method of Enriching One or More Species of Nucleic Acid Molecules in a Sample

In a third aspect, the present invention provides a method of enriching one or more species of nucleic acid molecules to which the unique 3′ sequences comprised by the nucleotide sequences of the multiple oligonucleotide species covalently attached to the particle of any one of the embodiments of the second aspect of the invention are at least 80% complementary in a sample, wherein the method comprises hybridization-based capture of the one or more species of nucleic acid molecules with the particle of any one of the embodiments of the second aspect of the invention.

In an embodiment, the nucleic acid molecules are RNA molecules or DNA molecules.

In an embodiment, the sample is selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries. In one such embodiment, the sample is selected from the group consisting of crude tissue lysates, cleared tissue lysates, crude cell lysates, and cleared cell lysates, the sample has been cross-linked, and the nucleic acid molecules have been cross-linked to one or more proteins and/or one or more other nucleic acid molecules.

In an embodiment, each of the unique 3′ sequences comprised by the nucleotide sequences of the multiple oligonucleotide species is complementary to a different stretch of the same species of nucleic acid molecules. In a different embodiment, each of the unique 3′ sequences comprised by the nucleotide sequences of the multiple oligonucleotide species is complementary to a different species of nucleic acid molecule.

### Fourth Aspect: Method of Depleting One or More Species of Nucleic Acid Molecules from a Sample

In a fourth aspect, the present invention provides a method of depleting one or more species of nucleic acid molecules to which the unique 3′ sequences comprised by the nucleotide sequences of the multiple oligonucleotide species covalently attached to the particle of any one of the embodiments of the second aspect of the invention are at least 80% complementary from a sample, wherein the method comprises hybridization-based capture of the one or more species of nucleic acid molecules with the particle of any one of the embodiments of the second aspect of the invention.

In an embodiment, the nucleic acid molecules are RNA molecules or DNA molecules.

In an embodiment, the sample is selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries. In one such embodiment, the sample is selected from the group consisting of crude tissue lysates, cleared tissue lysates, crude cell lysates, and cleared cell lysates, the sample has been cross-linked, and the nucleic acid molecules have been cross-linked to one or more proteins and/or one or more other nucleic acid molecules.

In an embodiment, each of the unique 3′ sequences comprised by the nucleotide sequences of the multiple oligonucleotide species is complementary to a different stretch of the same species of nucleic acid molecules. In a different embodiment, each of the unique 3′ sequences comprised by the nucleotide sequences of the multiple oligonucleotide species is complementary to a different species of nucleic acid molecule.

### BRIEF DESCRIPTION OF THE DRAWINGS

The figures shown in the following are merely illustrative and shall describe the present invention in a further way. These figures shall not be construed to limit the present invention thereto.

FIG. 1 Schematic drawing of step a) of the inventive method of producing a surface (depicted as a particle) on which multiple copies of each of multiple DNA oligonucleotide species are covalently attached at their 5′ ends. Dark grey sections of the free DNA oligonucleotide species represent their 3′ sequences that are complementary to sequence of the initial DNA oligonucleotides covalently attached to the surface and are identical between all free oligonucleotide species present. Light grey sections of the free DNA oligonucleotide species represent their 5′ sequences that differ between the different free oligonucleotide species such that they are unique to each free oligonucleotide species.

FIG. 2 Schematic drawing of step c) of the inventive method of producing a surface (depicted as a particle) on which multiple copies of each of multiple DNA oligonucleotide species are covalently attached at their 5′ ends. A. 3′ sequences of the free DNA oligonucleotide species (dark grey sections) have hybridized to the initial DNA oligonucleotides covalently attached to the surface (black) to form a DNA duplex, to which DNA-dependent DNA polymerase molecules have bound. Arrows indicate the direction in which elongation will proceed from the 3′ end of the initial DNA oligonucleotides. B. The initial oligonucleotides have been elongated by the DNA-dependent DNA-polymerase using the unique 5′ sequence (light grey) of the hybridized, formerly free, DNA oligonucleotide species as a template (see new medium grey section now covalently attached to the initial oligonucleotides).

FIG. 3 Schematic drawing of the end result (after step e)) of the inventive method of producing a surface (depicted as a particle) on which multiple copies of each of multiple DNA oligonucleotide species are covalently attached at their 5′ ends: the inventive particle on the surface of which multiple copies of each of multiple DNA oligonucleotide species are covalently attached. The 5′ sequence (black) is covalently attached to the particle, and the 3′ sequence (medium grey), differs between the different covalently attached DNA oligonucleotides species such that it is unique for each of the covalently attached DNA oligonucleotide species.

FIG. 4 Graphs representing the enrichment ratios of GAPDH and MALAT1 transcripts over ACTB and 18S RNA transcripts in enriched samples over input samples from experiments performed on RNA isolated from HEK293 cells.

FIG. 5 Graphs representing the enrichment ratios of GAPDH and MALAT1 transcripts over ACTB and 18S RNA transcripts in enriched samples over input samples from experiments performed on HEK293 cellular lysates.

### DEFINITIONS

For the sake of clarity and readability the following definitions are provided. Any technical feature mentioned for these definitions may be read on each and every embodiment of the invention. Additional definitions and explanations may be specifically provided in the context of these embodiments.

As used in the specification and the claims, the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

The term “surface” as used herein means the surface of a solid body. The body can be immobilized or non-immobilized. Exemplary surfaces are surfaces of, e.g., immobile plastic surfaces such as, e.g., DNA microarrays, and metal nanoparticles, such as magnetic nanoparticles and nanoparticles containing noble metals such as, e.g., gold nanoparticles, silver nanoparticles, and platinum nanoparticles. The term “particle” refers to a mobile solid body of a relatively small size, such that it can, e.g., move in a solution or liquid composition. For example, particles may be 1-10 micrometers in diameter.

The term “nucleic acid” means any DNA or RNA molecule and is used synonymously with polynucleotide. An “oligonucleotide” is a polynucleotide of a defined length, usually of a length of about 5 to about 1000 nucleotides, but not limited thereto. The specific order of the monomers, i.e. the order of the bases linked to the sugar/phosphate-backbone, is called the nucleotide sequence.

The term “DNA” is the usual abbreviation for “deoxyribonucleic acid”. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers or analogs thereof which are—by themselves—composed of a sugar moiety (deoxyribose), a base moiety and a phosphate moiety, and polymerize by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by NT-base-pairing and G/C-base-pairing.

The term “RNA” is the usual abbreviation for ribonucleic acid. It is a nucleic acid molecule, i.e. a polymer consisting of nucleotide monomers. These nucleotides are usually adenosine-monophosphate, uridine-monophosphate, guanosine-monophosphate and cytidine-monophosphate monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. The term “RNA” generally refers to a molecule or to a molecule species selected from the group consisting of long-chain RNA, coding RNA, non-coding RNA, single stranded RNA (ssRNA), double stranded RNA (dsRNA), linear RNA (linRNA), circular RNA (circRNA), messenger RNA (mRNA), RNA oligonucleotides, small interfering RNA (siRNA), small hairpin RNA (shRNA), antisense RNA (asRNA), CRISPR/Cas9 guide RNAs, riboswitches, immunostimulating RNA (isRNA), ribozymes, aptamers, ribosomal RNA (rRNA), transfer RNA (tRNA), viral RNA (vRNA), retroviral RNA or replicon RNA, small nuclear RNA (snRNA), small nucleolar RNA (snoRNA), microRNA (miRNA), circular RNA (circRNA), and a Piwi-interacting RNA (piRNA).

Both DNA and RNA may also contain modified nucleotides. The term “modified nucleotides” as used herein will be recognized and understood by the person of ordinary skill in the art, and is for example intended to comprise nucleotides that comprise a modification. For example, any nucleotide different from G, C, U, T, A may be regarded as a “modified nucleotide”. Modified nucleotides known in the art comprise 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-d methyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine, pseudouridine, N1-methylpseudouridine, N1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 2′-O-methyl uridine, pseudouridine (ψ), N1-methylpseudouridine (m1ψ), 5-methylcytosine, and 5-methoxyuridine.

The term “oligonucleotide species” as used herein means a defined oligonucleotide consisting of a predetermined nucleotide sequence. All members of a given oligonucleotide species have this same predetermined nucleotide sequence, i.e. are identical copies of each other.

The term “predetermined nucleotide sequence” as used herein means that each nucleotide at each position of the nucleotide sequence is known, i.e. is not random.

The term “unique for each oligonucleotide species” as used herein means that the unique nucleotide sequence stretch occurs only within one of the oligonucleotide species, and not in any other of the nucleotide species.

The term “initial oligonucleotide” as used herein means an oligonucleotide of a predetermined sequence that is covalently attached to a surface at their 5′ ends before the inventive method of producing a surface on which multiple copies of each of multiple DNA oligonucleotide species are covalently attached is performed, i.e. an oligonucleotide that was attached to a surface prior to the inventive method. An example of how this prior covalent attachment can be achieved is through direct synthesis of the oligonucleotide on the bead surface and was previously described in U.S. Pat. No. 5,512,439 A1. For the purposes of the present invention, the initial oligonucleotide is typically attached to the surface at its 5′ end.

The term “DNA-dependent DNA polymerase” as used herein means a polymerase that uses DNA as a template for elongating DNA. DNA-dependent DNA polymerases include polymerases that generate blunt ends, i.e. double-stranded DNA in which each strand has the same number of nucleotides, and polymerases that generate sticky ends or DNA overhangs, i.e. double-stranded DNA in which one strand is shorter than the other strand so that one or more bases at the end of the longer strand is/are not base-paired.

Examples of suitable DNA-dependent DNA polymerase are, e.g., Taq, Q5, Phusion, Bst, Bsu, phi29, T7, T4, KOD, SuperFi, Phire, Pfu, Tth, Pwo, DNA Polymerase I (E. coli), SD, (Following are reverse transcriptases): M-MuLV, AMV, WarmStart, rMoMuLV, SuperScript, SuperScript II, SuperScript III, Superscript IV, TGIRT. Examples of preferred DNA-dependent DNA polymerases are, e.g., Taq, Q5, and Phusion.

The term “RNA-dependent DNA polymerase” as used herein means a polymerase that uses RNA as a template for elongating DNA. Examples of suitable RNA-dependent DNA polymerase are, e.g., M-MuLV, AMV, WarmStart, rMoMuLV, SuperScript, SuperScript II, SuperScript III, Superscript IV, TGIRT.

The term “reaction buffer” as used herein means a weak acid or base used to maintain acidity (pH) of a reaction solution near a chosen value after the addition of another acid or base. Hence, the function of a buffer substance is to prevent rapid change in pH when acids or bases are added to the reaction solution.

The term “hybridization” as used herein refers to a single stranded DNA or RNA molecule with a specific sequence annealing to a complement sequence of a DNA or RNA molecule. Single stranded DNA can also hybridize with single stranded RNA to result in a DNA/RNA hybrid. Usually, a double-stranded DNA or RNA or a hybrid is stable under physiological conditions. An increase in temperature will usually cause the two hybridized or annealed strands to separate into single strands. A decrease in temperature causes the single stranded DNA and/or RNA molecules to anneal or hybridize to each other. Hybridization involves the formation of base pairs between A and T (or U) nucleotides and G and C nucleotides of the specific sequence and the complement sequence. “Hybridization” is usually carried out under stringent conditions, preferably under high stringency conditions. The term “high stringency conditions” is to be understood such that a specific sequence specifically hybridizes to a complement sequence in an amount that is detectably stronger than non-specific hybridization. High stringency conditions include conditions which distinguish an oligonucleotide with an exact complement sequence, or an oligonucleotide containing only a few mismatched nucleotides (e.g. 1, 2, 3, 4 or 5 mismatched nucleotides), from a random sequence that happens to have a few small complement regions (comprised of e.g. 3 to 4 nucleotides) to the specific sequence. Such small regions of complementarity melt more easily than a longer complement sequence of preferably about 10 to about 25 nucleotides, and high stringency hybridization makes them easily distinguishable. Relatively high stringency conditions include, for example, low salt and/or high temperature conditions, such as provided by about 0.02-0.1 M NaCl or the equivalent, at temperatures of about 50° C. to about 70° C. Such high stringency conditions tolerate little, if any, mismatch between a specific sequence and a complement sequence. It is generally appreciated that conditions can be rendered more stringent by the addition of increasing amounts of formamide.

The term “hybridization-based capture” as used herein means the capture of a nucleic acid molecule by sequence-specific hybridization with one or more wholly or partially complementary sequences comprised by one or more, respectively, oligonucleotide(s) that is/are covalently bound to the surface of a particle.

The term “nucleic acid elongation” as used herein means the addition of nucleotide monomers to an oligonucleotide in a template sequence-dependent manner, and may be performed, e.g., by a DNA polymerase, such as, e.g. a DNA-dependent DNA polymerase, or by an RNA-dependent DNA polymerase, such as, e.g., reverse transcriptase.

The term “complementary” means that a specific predetermined nucleotide sequence is either completely (which may be preferred) or in most parts the complement sequence of an underlying nucleotide sequence, such as, e.g. the sequence of a nucleic acid molecule to be captured, of an initial oligonucleotide, or of a free oligonucleotide species. Thus, put in other words, a complementary sequence is either 100% identical (which may be preferred) or is identical to a high degree to the complement sequence of the underlying sequence. When a nucleotide sequence is referred to as complementary, it is meant that it is complementary to such a degree that hybridization will take place specifically between it and its complement sequence. Accordingly, the complementary sequence is complementary to its complement sequence to such a degree that no hybridization between it and a non-complementary sequence takes place. It is generally preferred that the complement sequence of the oligonucleotide is 100% identical to the complement sequence of the underlying target sequence. When intending to mean less than 100% complementarity, the term “complementary” will be qualified herein with a preceding percentage.

The term “sequence identity” as used herein means that two nucleotide sequences are identical if they exhibit the same length and order of nucleotides. The percentage of identity typically describes the extent to which two sequences are identical, i.e. it typically describes the percentage of nucleotides that correspond in their sequence position to identical nucleotides of a reference sequence. For the determination of the degree of identity, the sequences to be compared are considered to exhibit the same length, i.e. the length of the longest sequence of the sequences to be compared. This means that a first sequence consisting of 8 nucleotides is 80% identical to a second sequence consisting of 10 nucleotides comprising the complete first sequence. In other words, in the context of the present invention, identity of sequences preferably relates to the percentage of nucleotides of a sequence, which have the same position in two sequences having the same length.

The term “denaturing” as used herein refers to applying conditions which interfere with or destroy non-covalent chemical bonds, such as e.g. base-pairing, leading to the loss of quaternary structure, tertiary structure, and secondary structure present in proteins or nucleic acids. Accordingly, denaturing nucleic acids will result in single-stranded nucleic acid strands without structure. Denaturing proteins will result in loss of folding and dissociation of any non-covalently linked subunits. Denaturation can be achieved by application of external stress, such as e.g. radiation or heat, or compounds such as, e.g., a strong acid or base, a concentrated inorganic salt, an organic solvent (e.g., alcohol or chloroform). Where the inventive method of producing a surface on which multiple copies of each of multiple DNA oligonucleotide species are covalently attached is concerned, denaturation of the polymerase-DNA complex preferably is achieved by application of heat, i.e. the third temperature is a relatively high temperature that is higher than the first and second temperatures. Preferably, such a denaturing temperature is from 90 to 98 degrees Celsius.

The term “separating” as used herein means the physical removal of one thing from another, e.g. of a surface from a denatured reaction mixture. Similarly, a captured nucleic acid molecule is separated from a sample by physically removing it from the sample or by physically removing other components of the sample from the nucleic acid molecule. Separation can occur, e.g., by removing a solution, sample, or reaction mixture from an immobilized surface, or by removing particles from a solution, sample, or reaction mixture. When particles are used and these particles are magnetic, e.g. paramagnetic, particles, separating is preferably achieved by magnetic separation, i.e. by application of magnetic forces that will attract the (para)magnetic particles but not the solution, sample, or reaction mixture.

The term “reaction mixture” as used herein refers to the components of a chemical or biochemical reaction within an appropriate buffer in which the reaction can occur.

The term “enriching” as used herein refers to an elevation of the concentration of the molecule to be enriched within a sample, solution, or reaction mixture, e.g. by removing other components of the sample, solution, or reaction mixture without removing the molecule to be enriched. In turn, the term “depleting” as used herein refers to a reduction of the concentration of the molecule to be depleted from a sample, solution, or reaction mixture, e.g. by removing the molecule to be depleted while not removing other components of the sample, solution, or reaction mixture.

The term “sample” as used herein means any sample in which one or more nucleic acid molecules are comprised. Samples include, e.g., partially isolated nucleic acids, isolated nucleic acids, biological samples, crude tissue lysates, cleared tissue lysates, crude cell lysates, cleared cell lysates, and processed and amplified nucleic acid sequencing libraries. The sample may also be, e.g., a crude tissue lysate, cleared tissue lysate, crude cell lysate, or cleared cell lysate, that has been cross-linked, i.e., wherein the nucleic acid molecules within the sample have been cross-linked to one or more proteins and/or one or more other nucleic acid molecules within the sample. “Cross-linking” refers to the covalent or ionic linkage of polymers (e.g. nucleic acid molecules, proteins). Cross-linking can be achieved, e.g., by chemical or ultraviolet light means.

### DETAILED DESCRIPTION OF THE INVENTION

The invention will be described in more detail in the following.

### First Aspect: Production of a Surface on which Multiple Copies of Each of Multiple DNA Oligonucleotide Species are Covalently Attached

In a first aspect, the present invention provides a method of producing a surface on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
f. providing
   i. a surface on which multiple copies of an initial DNA oligonucleotide are covalently attached at their 5′ ends, wherein the initial oligonucleotide has a predetermined nucleotide sequence;
   ii. a DNA-dependent DNA polymerase;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the surface, and
      a 5′ sequence that is unique for each of the multiple free oligonucleotide species;
g. hybridizing the multiple copies of the multiple free oligonucleotide species to the multiple copies of the initial oligonucleotide that is covalently attached to the surface at a first temperature at which a DNA duplex between the sequence of one copy of the covalently attached initial oligonucleotide and the complementary 3′ sequence of one copy of one of the free oligonucleotide species can form for each of the free oligonucleotide species;
h. elongating the multiple copies of the initial oligonucleotide that is covalently attached to the surface at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
i. denaturing the polymerase-DNA complex and duplex formed in step c at a third temperature; and
j. separating the surface from the denatured reaction mixture of step d.

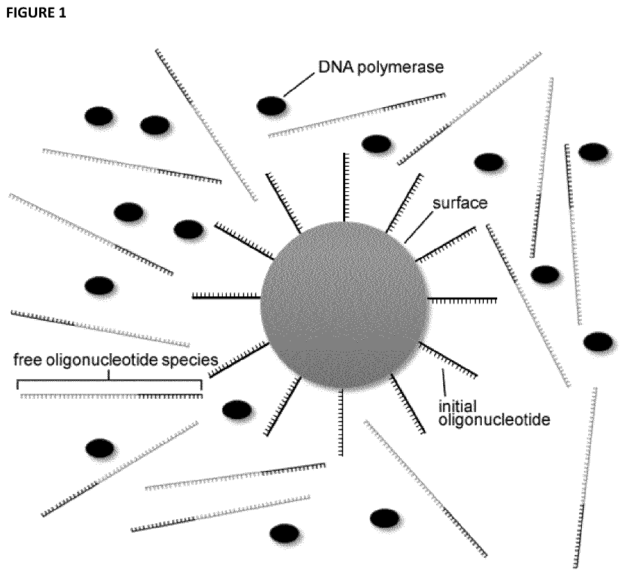

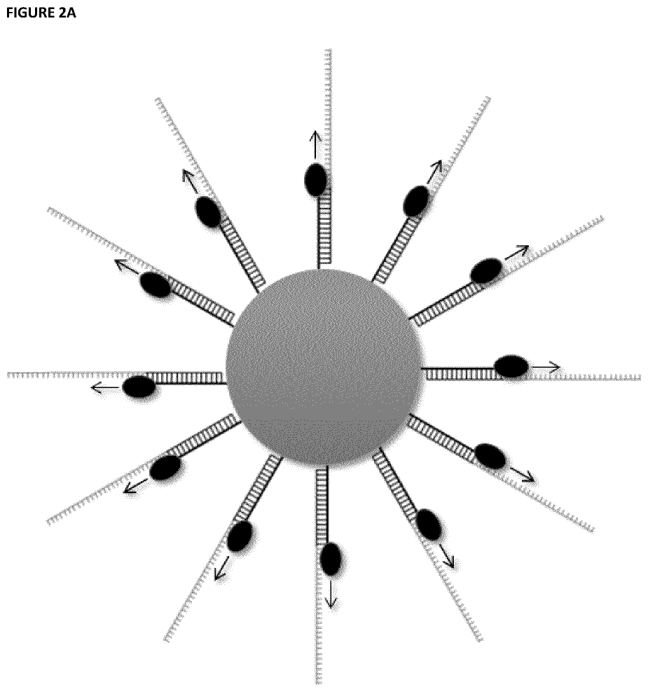

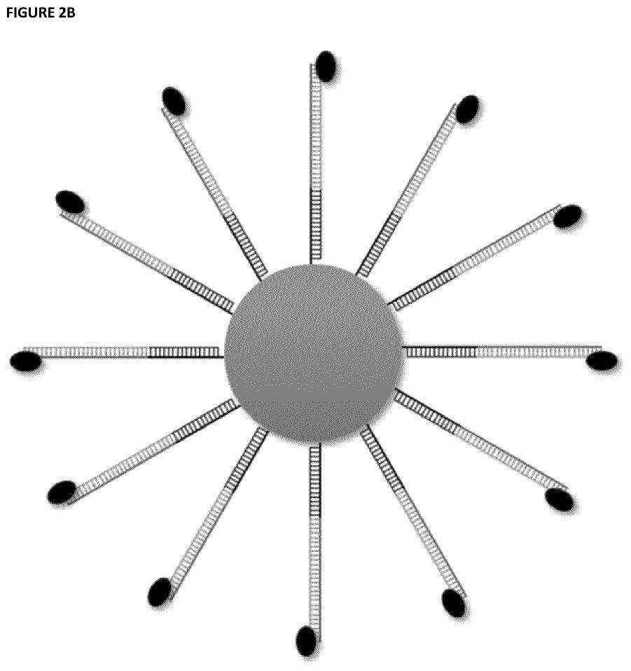

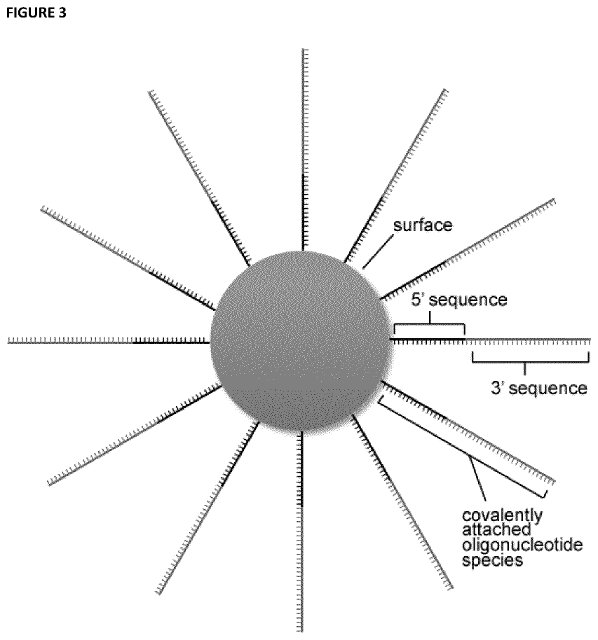

This process is schematically illustrated in FIGS. 1-3. As shown in FIG. 3, the end result of the inventive method is a surface on which multiple copies of each of multiple DNA oligonucleotide species are covalently attached at their 5′ ends wherein the DNA oligonucleotide species each have a predetermined nucleotide sequence comprising a 3′ sequence arising from the free DNA oligonucleotide species that is unique for each DNA oligonucleotide species and a 5′ sequence arising from the initial DNA oligonucleotide that is identical for each oligonucleotide species.

Importantly, this method allows for the simultaenous covalent attachment of multiple copies of each of multiple DNA oligonucleotide species in a shared reaction space, rather than to attach one DNA oligonucleotide species at a time in separate reaction spaces for each DNA oligonucleotide species.

In an embodiment, the surface is the surface of a particle. That is, the present invention provides a method of producing a particle on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a particle on which multiple copies of an initial DNA oligonucleotide are covalently attached at their 5′ ends, wherein the initial oligonucleotide has a predetermined nucleotide sequence;
   ii. a DNA-dependent DNA polymerase;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the particle, and
      a 5′ sequence that is unique for each of the multiple free oligonucleotide species;
b. hybridizing the multiple copies of the multiple free oligonucleotide species to the multiple copies of the initial oligonucleotide that is covalently attached to the particle at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the particle at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and
e. separating the particle from the denatured reaction mixture of step d.

In a preferred embodiment, the particle is a magnetic particle. That is, the present invention provides a method of producing a magnetic particle on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a magnetic particle on which multiple copies of an initial DNA oligonucleotide are covalently attached at their 5′ ends, wherein the initial oligonucleotide has a predetermined nucleotide sequence;
   ii. a DNA-dependent DNA polymerase;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the magnetic particle, and
      a 5′ sequence that is unique for each of the multiple free oligonucleotide species;
b. hybridizing the multiple copies of the multiple free oligonucleotide species to the multiple copies of the initial oligonucleotide that is covalently attached to the magnetic particle at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the magnetic particle at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step c at a third temperature; and
e. separating the magnetic particle from the denatured reaction mixture of step d.

In a more preferred embodiment, the magnetic particle is a paramagnetic particle, and the separating in step e is magnetically separating. That is, the present invention provides a method of producing a paramagnetic particle on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a paramagnetic particle on which multiple copies of an initial DNA oligonucleotide are covalently attached at their 5′ ends, wherein the initial oligonucleotide has a predetermined nucleotide sequence;
   ii. a DNA-dependent DNA polymerase;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the paramagnetic particle, and
      a 5′ sequence that is unique for each of the multiple free oligonucleotide species;
b. hybridizing the multiple copies of the multiple free oligonucleotide species to the multiple copies of the initial oligonucleotide that is covalently attached to the paramagnetic particle at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the paramagnetic particle at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and
e. magnetically separating the paramagnetic particle from the denatured reaction mixture of step d.

In an embodiment, the DNA-dependent DNA polymerase is selected from the group consisting of DNA-dependent DNA polymerase that produce blunt ends and DNA-dependent DNA polymerase that produce sticky ends. That is, the present invention provides a method of producing a surface on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a surface on which multiple copies of an initial DNA oligonucleotide are covalently attached at their 5′ ends, wherein the initial oligonucleotide has a predetermined nucleotide sequence;
   ii. a DNA-dependent DNA polymerase selected from the group consisting of DNA-dependent DNA polymerase that produce blunt ends and DNA-dependent DNA polymerase that produce sticky ends;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the surface, and
      a 5′ sequence that is unique for each of the multiple free oligonucleotide species;
b. hybridizing the multiple copies of the multiple free oligonucleotide species to the multiple copies of the initial oligonucleotide that is covalently attached to the surface at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the surface at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and
e. separating the surface from the denatured reaction mixture of step d.

In an embodiment, the surface is the surface of a particle. That is, the present invention provides a method of producing a particle on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a particle on which multiple copies of an initial DNA oligonucleotide are covalently attached at their 5′ ends, wherein the initial oligonucleotide has a predetermined nucleotide sequence;
   ii. a DNA-dependent DNA polymerase selected from the group consisting of DNA-dependent DNA polymerase that produce blunt ends and DNA-dependent DNA polymerase that produce sticky ends;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the particle, and
      a 5′ sequence that is unique for each of the multiple free oligonucleotide species;
b. hybridizing the multiple copies of the multiple free oligonucleotide species to the multiple copies of the initial oligonucleotide that is covalently attached to the particle at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the particle at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and
e. separating the particle from the denatured reaction mixture of step d.

In a preferred embodiment, the particle is a magnetic particle. That is, the present invention provides a method of producing a magnetic particle on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a magnetic particle on which multiple copies of an initial DNA oligonucleotide are covalently attached at their 5′ ends, wherein the initial oligonucleotide has a predetermined nucleotide sequence;
   ii. a DNA-dependent DNA polymerase selected from the group consisting of DNA-dependent DNA polymerase that produce blunt ends and DNA-dependent DNA polymerase that produce sticky ends;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the magnetic particle, and
      a 5′ sequence that is unique for each of the multiple free oligonucleotide species;
b. hybridizing the multiple copies of the multiple free oligonucleotide species to the multiple copies of the initial oligonucleotide that is covalently attached to the magnetic particle at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the magnetic particle at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and
e. separating the magnetic particle from the denatured reaction mixture of step d.

In a more preferred embodiment, the magnetic particle is a paramagnetic particle, and the separating in step e is magnetically separating. That is, the present invention provides a method of producing a paramagnetic particle on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
f. providing
   i. a paramagnetic particle on which multiple copies of an initial DNA oligonucleotide are covalently attached at their 5′ ends, wherein the initial oligonucleotide has a predetermined nucleotide sequence;
   ii. a DNA-dependent DNA polymerase selected from the group consisting of DNA-dependent DNA polymerase that produce blunt ends and DNA-dependent DNA polymerase that produce sticky ends;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the paramagnetic particle, and
      a 5′ sequence that is unique for each of the multiple free oligonucleotide species;
g. hybridizing the multiple copies of the multiple free oligonucleotide species to the multiple copies of the initial oligonucleotide that is covalently attached to the paramagnetic particle at a first temperature at which a DNA duplex between the sequence of one copy of the covalently attached initial oligonucleotide and the complementary 3′ sequence of one copy of one of the free oligonucleotide species can form for each of the free oligonucleotide species;
h. elongating the multiple copies of the initial oligonucleotide that is covalently attached to the paramagnetic particle at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
i. denaturing the polymerase-DNA complex and duplex formed in step c at a third temperature; and magnetically separating the paramagnetic particle from the denatured reaction mixture of step d.

In a preferred embodiment, the DNA-dependent DNA polymerase is a DNA-dependent DNA polymerase that produces blunt ends. That is, the present invention provides a method of producing a surface on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a surface on which multiple copies of an initial DNA oligonucleotide are covalently attached at their 5′ ends, wherein the initial oligonucleotide has a predetermined nucleotide sequence;
   ii. a DNA-dependent DNA polymerase that produce blunt ends;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the surface, and
      a 5′ sequence that is unique for each of the multiple free oligonucleotide species;
b. hybridizing the multiple copies of the multiple free oligonucleotide species to the multiple copies of the initial oligonucleotide that is covalently attached to the surface at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the surface at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and
e. separating the surface from the denatured reaction mixture of step d.

In an embodiment, the surface is the surface of a particle. That is, the present invention provides a method of producing a particle on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a particle on which multiple copies of an initial DNA oligonucleotide are covalently attached at their 5′ ends, wherein the initial oligonucleotide has a predetermined nucleotide sequence;
   ii. a DNA-dependent DNA polymerase that produce blunt ends;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the particle, and
      a 5′ sequence that is unique for each of the multiple free oligonucleotide species;
b. hybridizing the multiple copies of the multiple free oligonucleotide species to the multiple copies of the initial oligonucleotide that is covalently attached to the particle at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the particle at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and
e. separating the particle from the denatured reaction mixture of step d.

In a preferred embodiment, the particle is a magnetic particle. That is, the present invention provides a method of producing a magnetic particle on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a magnetic particle on which multiple copies of an initial DNA oligonucleotide are covalently attached at their 5′ ends, wherein the initial oligonucleotide has a predetermined nucleotide sequence;
   ii. a DNA-dependent DNA polymerase that produce blunt ends;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the magnetic particle, and
      a 5′ sequence that is unique for each of the multiple free oligonucleotide species;
b. hybridizing the multiple copies of the multiple free oligonucleotide species to the multiple copies of the initial oligonucleotide that is covalently attached to the magnetic particle at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the magnetic particle at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and
e. separating the magnetic particle from the denatured reaction mixture of step d.

In a more preferred embodiment, the magnetic particle is a paramagnetic particle, and the separating in step e is magnetically separating. That is, the present invention provides a method of producing a paramagnetic particle on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising the following steps:
a. providing
   i. a paramagnetic particle on which multiple copies of an initial DNA oligonucleotide are covalently attached at their 5′ ends, wherein the initial oligonucleotide has a predetermined nucleotide sequence;
   ii. a DNA-dependent DNA polymerase that produce blunt ends;
   iii. deoxyribonucleotide triphosphates;
   iv. a reaction buffer suitable for DNA hybridization and elongation by the DNA-dependent DNA polymerase
   v. multiple copies of multiple free DNA oligonucleotide species, wherein the free oligonucleotide species each have a predetermined nucleotide sequence comprising
      a 3′ sequence that is complementary to a 3′ sequence of the nucleotide sequence of the initial oligonucleotide that is covalently attached to the paramagnetic particle, and
      a 5′ sequence that is unique for each of the multiple free oligonucleotide species;

b. hybridizing the multiple copies of the multiple free oligonucleotide species to the multiple copies of the initial oligonucleotide that is covalently attached to the paramagnetic particle at a first temperature at which a DNA duplex between the sequence of one copy of the covalently attached initial oligonucleotide and the complementary 3′ sequence of one copy of one of the free oligonucleotide species can form for each of the free oligonucleotide species;
c. elongating the multiple copies of the initial oligonucleotide that is covalently attached to the paramagnetic particle at a second temperature by means of the DNA-dependent DNA polymerase binding the duplex formed in step b, thereby forming a polymerase-DNA complex, and attaching the deoxyribonucleotide triphosphates to the 3′ end of the covalently attached initial oligonucleotide using the hybridized oligonucleotide species as a template;
d. denaturing the polymerase-DNA complex and duplex formed in step cat a third temperature; and magnetically separating the paramagnetic particle from the denatured reaction mixture of step d.

In an embodiment, in any one of the inventive production methods,
the first temperature is from 25° C. to 72° C., e.g. 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, or 72° C., and/or
the second temperature is from 40° C. to 78° C., e.g. 40, 41, 42, 43, 45, 46, 47, 48, 49, 50, 51, 52, 53, 54, 55, 56, 57, 58, 59, 60, 61, 62, 63, 64, 65, 66, 67, 68, 69 70, 71, 72, 73, 74, 75, 76, 77, or 78° C.,
   optionally from 60° C. to 78° C., e.g. 60, 61, 62, 63, 64, 65, 66, 67, 68, 69 70, 71, 72, 73, 74, 75, 76, 77, or 78° C., and/or
the third temperature is from 90° C. to 98° C., e.g. 90, 91, 92, 93, 94, 95, 96, 97, or 98° C.

That is, in an embodiment, in any one of the inventive production methods, the first temperature is from 25° C. to 72° C., e.g. 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, or 72° C. In an embodiment, the first temperature is from 25° C. to 60° C.

In an embodiment, in any one of the inventive production methods, the second temperature is from 40° C. to 78° C., e.g. 40, 41, 42, 43, 45, 46, 47, 48, 49, 50, 51, 52, 53, 54, 55, 56, 57, 58, 59, 60, 61, 62, 63, 64, 65, 66, 67, 68, 69 70, 71, 72, 73, 74, 75, 76, 77, or 78° C. In a preferred such embodiment, the second temperature is from 60° C. to 78° C., e.g. 60, 61, 62, 63, 64, 65, 66, 67, 68, 69 70, 71, 72, 73, 74, 75, 76, 77, or 78° C.

In an embodiment, in any one of the inventive production methods, the third temperature is from 90° C. to 98° C., e.g. 90, 91, 92, 93, 94, 95, 96, 97, or 98° C.

In an embodiment, in any one of the inventive production methods, the first temperature is from 25° C. to 72° C., e.g. 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, or 72° C., and the second temperature is from 40° C. to 78° C., e.g. 40, 41, 42, 43, 45, 46, 47, 48, 49, 50, 51, 52, 53, 54, 55, 56, 57, 58, 59, 60, 61, 62, 63, 64, 65, 66, 67, 68, 69 70, 71, 72, 73, 74, 75, 76, 77, or 78° C.

In a preferred embodiment, in any one of the inventive production methods, the first temperature is from 25° C. to 72° C., e.g. 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, or 72° C., and the second temperature is from 60° C. to 78° C., e.g. 60, 61, 62, 63, 64, 65, 66, 67, 68, 69 70, 71, 72, 73, 74, 75, 76, 77, or 78° C.

In an embodiment, in any one of the inventive production methods, the first temperature is from 25° C. to 72° C., e.g. 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, or 72° C., and third temperature is from 90° C. to 98° C., e.g. 90, 91, 92, 93, 94, 95, 96, 97, or 98° C.

In an embodiment, in any one of the inventive production methods, the first temperature is from 25° C. to 72° C., e.g. 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, or 72° C., the second temperature is from 40° C. to 78° C., e.g. 40, 41, 42, 43, 45, 46, 47, 48, 49, 50, 51, 52, 53, 54, 55, 56, 57, 58, 59, 60, 61, 62, 63, 64, 65, 66, 67, 68, 69 70, 71, 72, 73, 74, 75, 76, 77, or 78° C., and third temperature is from 90° C. to 98° C., e.g. 90, 91, 92, 93, 94, 95, 96, 97, or 98° C.

In a preferred embodiment, in any one of the inventive production methods, the first temperature is from 25° C. to 72° C., e.g. 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, or 72° C., the second temperature is from 60° C. to 78° C., e.g. 60, 61, 62, 63, 64, 65, 66, 67, 68, 69 70, 71, 72, 73, 74, 75, 76, 77, or 78° C., and third temperature is from 90° C. to 98° C., e.g. 90, 91, 92, 93, 94, 95, 96, 97, or 98° C.

In an embodiment, in any one of the inventive production methods, the second temperature is from 40° C. to 78° C., e.g. 40, 41, 42, 43, 45, 46, 47, 48, 49, 50, 51, 52, 53, 54, 55, 56, 57, 58, 59, 60, 61, 62, 63, 64, 65, 66, 67, 68, 69 70, 71, 72, 73, 74, 75, 76, 77, or 78° C., and third temperature is from 90° C. to 98° C., e.g. 90, 91, 92, 93, 94, 95, 96, 97, or 98° C.

In a preferred embodiment, in any one of the inventive production methods, the second temperature is from 60° C. to 78° C., e.g. 60, 61, 62, 63, 64, 65, 66, 67, 68, 69 70, 71, 72, 73, 74, 75, 76, 77, or 78° C., and third temperature is from 90° C. to 98° C., e.g. 90, 91, 92, 93, 94, 95, 96, 97, or 98° C.

In an embodiment, in any one of the inventive production methods, both the first and second temperatures are from 40° C. to 72° C., e.g. 40° C. to 60° C., e.g. 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, or 72° C., and steps b and c are performed concurrently.

In an embodiment, in any one of the inventive production methods, both the first and second temperatures are from 40° C. to 72° C., e.g. 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, or 72° C., the third temperature is from 90° C. to 98° C., e.g. 90, 91, 92, 93, 94, 95, 96, 97, or 98° C., and steps b and c are performed concurrently.

In an embodiment, in any one of the inventive production methods, the initial oligonucleotide that is covalently attached at its 5′ end to the surface is from 5 nucleotides to 100 nucleotides in length, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100 nucleotides. In a preferred embodiment, in any one of the inventive production methods, the initial oligonucleotide that is covalently attached to the surface is from 10 nucleotides to 20 nucleotides in length, e.g., 10, 11, 12, 13, 14, 15, 16, 17, 18, 19, 20 nucleotides.

In an embodiment, the free oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides. In a preferred embodiment, in any one of the inventive production methods, the free oligonucleotide species are from 24 nucleotides to 50 nucleotides, in length, e.g. 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In an embodiment, in any one of the inventive production methods, the initial oligonucleotide that is covalently attached at its 5′ end to the surface is from 5 nucleotides to 100 nucleotides in length, e.g. 5, 6, 7, 8, 9, 10, 11, 12, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100 nucleotides, and the free oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides.

In a preferred embodiment, in any one of the inventive production methods, the initial oligonucleotide that is covalently attached to the surface is from 10 nucleotides to 20 nucleotides in length, e.g., 10, 11, 12, 13, 14, 15, 16, 17, 18, 19, 20 nucleotides, and the free oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides.

In an embodiment, in any one of the inventive production methods, the 3′ sequence of the free oligonucleotide species that is complimentary to the 3′ sequence of the covalently attached initial oligonucleotide is from 5 nucleotides to 100 nucleotides in length, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100 nucleotides. In a preferred embodiment, in any one of the inventive production methods, the 3′ sequence of the free oligonucleotide species that is complimentary to the 3′ sequence of the covalently attached initial oligonucleotide is from 10 nucleotides to 20 nucleotides, in length, e.g., 10, 11, 12, 13, 14, 15, 16, 17, 18, 19, 20 nucleotides.

In an embodiment, in any one of the inventive production methods, the initial oligonucleotide that is covalently attached at its 5′ end to the surface is from 5 nucleotides to 100 nucleotides in length, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100 nucleotides, and the 3′ sequence of the free oligonucleotide species that is complimentary to the 3′ sequence of the covalently attached initial oligonucleotide is from 5 nucleotides to 100 nucleotides in length, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100 nucleotides.

In a preferred embodiment, in any one of the inventive production methods, the initial oligonucleotide that is covalently attached at its 5′ end to the surface is from 10 nucleotides to 20 nucleotides in length, e.g., 10, 11, 12, 13, 14, 15, 16, 17, 18, 19, 20 nucleotides, and the 3′ sequence of the free oligonucleotide species that is complimentary to the 3′ sequence of the covalently attached initial oligonucleotide is from 10 nucleotides to 20 nucleotides, in length, e.g., 10, 11, 12, 13, 14, 15, 16, 17, 18, 19, 20 nucleotides.

In an embodiment, in any one of the inventive production methods, the initial oligonucleotide that is covalently attached at its 5′ end to the surface is from 5 nucleotides to 100 nucleotides in length, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100 nucleotides, the free oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides, and the 3′ sequence of the free oligonucleotide species that is complimentary to the 3′ sequence of the covalently attached initial oligonucleotide is from 5 nucleotides to 100 nucleotides in length, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100 nucleotides.

In a preferred embodiment, in any one of the inventive production methods, the initial oligonucleotide that is covalently attached to the surface is from 10 nucleotides to 20 nucleotides in length, e.g., 10, 11, 12, 13, 14, 15, 16, 17, 18, 19, 20 nucleotides, and the free oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides, and the 3′ sequence of the free oligonucleotide species that is complimentary to the 3′ sequence of the covalently attached initial oligonucleotide is from 10 nucleotides to 20 nucleotides, in length, e.g., 10, 11, 12, 13, 14, 15, 16, 17, 18, 19, 20 nucleotides.

In any one of the inventive production methods, a proper design of the oligonucleotide sequences of both the initial DNA oligonucleotide and the multiple free DNA oligonucleotides should result in a situation in which the initial DNA oligonucleotide has a significantly lower temperature of hybridization to its respective complementary sequence than the unique 5′ sequences of the free DNA oligonucleotides species to their respective complementary sequences. The difference in melting temperatures (Tm) should be equal or higher than 10° C. This allows for the utilization of the resulting surfaces (which, when the surface is a particle, are provided by the second aspect of the present invention) in the hybridization based capture of nucleic acid molecules (which, when the surface is a particle, is provided by the third and fourth aspects of the present disclosure) under temperature conditions that eliminate unwanted hybridization of the 5′ sequence of the DNA oligonucleotide species attached to the surface (which 5′ sequence arises from the initial DNA oligonucleotide) to nucleic acid molecules, while at the same time promoting optimal hybridization of the unique 3′ sequences of the DNA oligonucleotide species attached to the surface (which 3′ sequence arises from the unique 5′ sequence of the free oligonucleotide species) to its target, the complementary sequence comprised by the nucleic acid molecule to be captured. Controlling for an appropriate difference is described in Example 2 below.

Furthermore, in any one of the inventive production methods, the unique 5′ sequence of the free DNA oligonucleotide species should not have any significant tendencies to form dimers within that 5′ sequence, between two copies of the respective DNA oligonucleotide species, or with the initial DNA oligonucleotide covalently attached to the surface at its 5′ end. The tendency to form these dimers can be controlled on the level of design by checking the levels of complementarity between the initial DNA oligonucleotide and the unique 5′ sequence of the free DNA oligonucleotide species and selecting sequences with a Tm of 10° C. or more below the Tm for dimerization between the initial DNA oligonucleotide and the unique 5′ sequence of the free DNA oligonucleotide species.

Preferably, in any one of the inventive production methods, the initial DNA oligonucleotide has a Tm of from 38° C. to 70° C., e.g. from 38° C. to 50° C., e.g. 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70° C. In one preferred embodiment, the Tm is 44.7° C.

Furthermore, in any one of the inventive production methods, the 5′ unique sequence of the free DNA oligonucleotide species should only be found in the nucleic acid molecule to be captured, e.g. for enrichment or depletion in/from a sample, but not in other nucleic acid molecules present in the sample.

In an alternative approach, in any one of the inventive production methods described herein, free RNA oligonucleotide species instead of free DNA oligonucleotide species and an RNA-dependent DNA-polymerase instead of a DNA-dependent DNA polymerase, e.g. reverse transcriptase, may be employed instead. The end result, i.e. the particles provided by the second aspect of the present invention, will remain unchanged.

### Second Aspect: Particles on the Surface of which Multiple Copies of Each of Multiple DNA Oligonucleotide Species are Covalently Attached at their 5′ Ends

In a second aspect, the present invention provides a particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species.

In a preferred embodiment, the particle is a magnetic particle. That is, the present invention provides a magnetic particle on the surface of which multiple copies of each of multiple DNA oligonucleotide species are covalently attached at their 5′ ends, wherein the oligonucleotide species each have a predetermined nucleotide sequence, and wherein the predetermined nucleotide sequence of each oligonucleotide species comprises a 3′ sequence that is unique for each of the oligonucleotide species.

In a more preferred embodiment, the magnetic particle is a paramagnetic particle. That is, the present invention provides a paramagnetic particle on the surface of which multiple copies of each of multiple DNA oligonucleotide species are covalently attached wherein the oligonucleotide species each have a predetermined nucleotide sequence, and wherein the predetermined nucleotide sequence of each oligonucleotide species comprises a 3′ sequence that is unique for each of the oligonucleotide species.

In an embodiment, the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides.

That is, the present invention provides a particle on the surface of which multiple copies of each of multiple DNA oligonucleotide species are covalently attached at their 5′ ends, wherein the oligonucleotide species each have a predetermined nucleotide sequence, and wherein the predetermined nucleotide sequence of each oligonucleotide species comprises a 3′ sequence that is unique for each of the DNA oligonucleotide species, and wherein the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides.

In a preferred embodiment, the particle is a magnetic particle. That is, the present invention provides a magnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides.

In a more preferred embodiment, the magnetic particle is a paramagnetic particle. That is, the present invention provides a paramagnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides.

In a preferred embodiment, the DNA oligonucleotide species are from 24 nucleotides to 70 nucleotides, e.g. 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, in length.

That is, the present invention provides a particle on the surface of which multiple copies of each of multiple DNA oligonucleotide species are covalently attached at their 5′ ends, wherein the oligonucleotide species each have a predetermined nucleotide sequence, and wherein the predetermined nucleotide sequence of each oligonucleotide species comprises a 3′ sequence that is unique for each of the DNA oligonucleotide species, and wherein the DNA oligonucleotide species are from 24 nucleotides to 70 nucleotides, e.g. 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, in length.

In a preferred embodiment, the particle is a magnetic particle. That is, the present invention provides a magnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 24 nucleotides to 70 nucleotides, e.g. 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, in length.

In a more preferred embodiment, the magnetic particle is a paramagnetic particle. That is, the present invention provides a paramagnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 24 nucleotides to 70 nucleotides, e.g. 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, in length.

In an embodiment, the unique 3′ sequence is from 5 nucleotides to 995 nucleotides, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 960, 970, 980, 985, 990, or 995 nucleotides, in length.

That is, the present invention provides a particle on the surface of which multiple copies of each of multiple DNA oligonucleotide species are covalently attached at their 5′ ends, wherein the oligonucleotide species each have a predetermined nucleotide sequence, wherein the predetermined nucleotide sequence of each oligonucleotide species comprises a 3′ sequence that is unique for each of the oligonucleotide species, and wherein the unique 3′ sequence is from 5 nucleotides to 995 nucleotides, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 960, 970, 980, 985, 990, or 995 nucleotides, in length.

In a preferred embodiment, the particle is a magnetic particle. That is, the present invention provides a magnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species and wherein the unique 3′ sequence is from 5 nucleotides to 995 nucleotides, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 960, 970, 980, 985, 990, or 995 nucleotides, in length.

In a more preferred embodiment, the magnetic particle is a paramagnetic particle. That is, the present invention provides a paramagnetic particle on the surface of which multiple copies of each of multiple DNA oligonucleotide species are covalently attached at their 5′ ends wherein the oligonucleotide species each have a predetermined nucleotide sequence, wherein the predetermined nucleotide sequence of each oligonucleotide species comprises a 3′ sequence that is unique for each of the oligonucleotide species, and wherein the unique 3′ sequence is from 5 nucleotides to 995 nucleotides, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 960, 970, 980, 985, 990, or 995 nucleotides, in length.

In an embodiment, the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides.

That is, the present invention provides a particle on the surface of which multiple copies of each of multiple DNA oligonucleotide species are covalently attached at their 5′ ends, wherein the oligonucleotide species each have a predetermined nucleotide sequence, and wherein the predetermined nucleotide sequence of each oligonucleotide species comprises a 3′ sequence that is unique for each of the DNA oligonucleotide species, and wherein the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides, and wherein the unique 3′ sequence is from 5 nucleotides to 995 nucleotides, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 960, 970, 980, 985, 990, or 995 nucleotides, in length.

In a preferred embodiment, the particle is a magnetic particle. That is, the present invention provides a magnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides, and wherein the unique 3′ sequence is from 5 nucleotides to 995 nucleotides, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 960, 970, 980, 985, 990, or 995 nucleotides, in length.

In a more preferred embodiment, the magnetic particle is a paramagnetic particle. That is, the present invention provides a paramagnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides, and wherein the unique 3′ sequence is from 5 nucleotides to 995 nucleotides, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 960, 970, 980, 985, 990, or 995 nucleotides, in length.

In a preferred embodiment, the unique 3′ sequence is from 12 nucleotides to 50 nucleotides, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, in length.

That is, the present invention provides a particle on the surface of which multiple copies of each of multiple DNA oligonucleotide species are covalently attached at their 5′ ends, wherein the oligonucleotide species each have a predetermined nucleotide sequence, wherein the predetermined nucleotide sequence of each oligonucleotide species comprises a 3′ sequence that is unique for each of the oligonucleotide species, and wherein the unique 3′ sequence is from 12 nucleotides to 50 nucleotides, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, in length.

In a preferred embodiment, the particle is a magnetic particle. That is, the present invention provides a magnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species and wherein the unique 3′ sequence is from 12 nucleotides to 50 nucleotides, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, in length.

In a more preferred embodiment, the magnetic particle is a paramagnetic particle. That is, the present invention provides a paramagnetic particle on the surface of which multiple copies of each of multiple DNA oligonucleotide species are covalently attached at their 5′ ends wherein the oligonucleotide species each have a predetermined nucleotide sequence, wherein the predetermined nucleotide sequence of each oligonucleotide species comprises a 3′ sequence that is unique for each of the oligonucleotide species, and wherein the unique 3′ sequence is from 12 nucleotides to 50 nucleotides, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, in length.

In an embodiment, the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides.

That is, the present invention provides a particle on the surface of which multiple copies of each of multiple DNA oligonucleotide species are covalently attached at their 5′ ends, wherein the oligonucleotide species each have a predetermined nucleotide sequence, and wherein the predetermined nucleotide sequence of each oligonucleotide species comprises a 3′ sequence that is unique for each of the DNA oligonucleotide species, and wherein the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides, and wherein the unique 3′ sequence is from 12 nucleotides to 50 nucleotides, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, in length.

In a preferred embodiment, the particle is a magnetic particle. That is, the present invention provides a magnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides, and wherein the unique 3′ sequence is from 12 nucleotides to 50 nucleotides, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, in length.

In a more preferred embodiment, the magnetic particle is a paramagnetic particle. That is, the present invention provides a paramagnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 10 nucleotides to 1000 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 nucleotides, and wherein the unique 3′ sequence is from 12 nucleotides to 50 nucleotides, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, in length.

In a preferred embodiment, the DNA oligonucleotide species are from 24 nucleotides to 70 nucleotides, e.g. 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, in length.

That is, the present invention provides a particle on the surface of which multiple copies of each of multiple DNA oligonucleotide species are covalently attached at their 5′ ends, wherein the oligonucleotide species each have a predetermined nucleotide sequence, and wherein the predetermined nucleotide sequence of each oligonucleotide species comprises a 3′ sequence that is unique for each of the DNA oligonucleotide species, and wherein the DNA oligonucleotide species are from 24 nucleotides to 70 nucleotides, e.g. 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, in length, and wherein the unique 3′ sequence is from 12 nucleotides to 50 nucleotides, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, in length.

In a preferred embodiment, the particle is a magnetic particle. That is, the present invention provides a magnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 24 nucleotides to 70 nucleotides, e.g. 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, in length and wherein the unique 3′ sequence is from 12 nucleotides to 50 nucleotides, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, in length.

In a more preferred embodiment, the magnetic particle is a paramagnetic particle. That is, the present invention provides a paramagnetic particle on the surface of which multiple copies of each of multiple DNA oligonucleotide species are covalently attached at their 5′ ends wherein the oligonucleotide species each have a predetermined nucleotide sequence, and wherein the predetermined nucleotide sequence of each oligonucleotide species comprises a 3′ sequence that is unique for each of the oligonucleotide species, and wherein the DNA oligonucleotide species are from 24 nucleotides to 70 nucleotides, e.g. 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, in length.

For any one of the inventive particles, the 5′ sequence of the DNA oligonucleotide species covalently attached at their 5′ ends to the particle should have a significantly lower temperature of hybridization to its respective complementary sequence than the unique 3′ sequences of the DNA oligonucleotide species covalently attached to the particle to their respective complementary sequences. The difference in melting temperatures (Tm) should be equal or higher than 10° C. This allows for the utilization of the inventive particle in the hybridization based capture of nucleic acid molecules (which is provided by the third and fourth aspects of the present disclosure) under temperature conditions that eliminate unwanted hybridization of the 5′ sequence of the DNA oligonucleotide species attached to the surface to complementary sequences comprised by nucleic acid molecules, while at the same time promoting optimal hybridization of the unique 3′ sequences of the DNA oligonucleotide species attached to the surface to their respective target, the respective complementary sequence comprised by the nucleic acid molecule to be captured. Controlling for an appropriate difference is described in Example 2 below.

Preferably, for any of inventive particles, the 5′ sequence of the DNA oligonucleotide species covalently attached at their 5′ ends to the particle has a Tm of from 38° C. to 70° C., e.g. from 38° C. to 50° C., e.g. 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or 71° C. In one preferred embodiment, the Tm is 44.7° C.

In an embodiment, the multiple DNA oligonucleotide species of any of the inventive particles do not comprise a chemical 3′ modification, such as, e.g. dideoxy nucleoside triphosphate (ddNTP), inverted nucleoside triphosphate, Spacer C3 (Sp3), Spacer C6 (Sp6), Spacer C12 (SpC12).

### Third Aspect: Method of Enriching One or More Species of Nucleic Acid Molecules in a Sample

In a third aspect, the present invention provides a method of enriching one or more species of nucleic acid molecules to which the unique 3′ sequences comprised by the nucleotide sequences of the multiple oligonucleotide species covalently attached at their 5′ ends to the particle of any one of the embodiments of the second aspect of the invention are at least 80%, e.g. at least 80, 81, 82, 83, 84, 85, 86, 87, 88, 89, 90, 91, 92, 93, 94, 95, 96, 97, 98, 99, or 99.5%, or 100%, complementary in a sample, wherein the method comprises hybridization-based capture of the one or more species of nucleic acid molecules with the particle of any one of the embodiments of the second aspect of the invention.

In an embodiment, the nucleic acid molecules are RNA molecules or DNA molecules. That is, the present invention provides a method of enriching one or more species of RNA molecules to which the unique 3′ sequences comprised by the nucleotide sequences of the multiple oligonucleotide species covalently attached at their 5′ ends to the particle of any one of the embodiments of the second aspect of the invention are at least 80%, e.g. at least 80, 81, 82, 83, 84, 85, 86, 87, 88, 89, 90, 91, 92, 93, 94, 95, 96, 97, 98, 99, or 99.5%, or 100%, complementary in a sample, wherein the method comprises hybridization-based capture of the one or more species of RNA molecules with the particle of any one of the embodiments of the second aspect of the invention. Further, the present invention provides a method of enriching one or more species of DNA molecules to which the unique 3′ sequences comprised by the nucleotide sequences of the multiple oligonucleotide species covalently attached at their 5′ ends to the particle of any one of the embodiments of the second aspect of the invention are at least 80% complementary in a sample, wherein the method comprises hybridization-based capture of the one or more species of DNA molecules with the particle of any one of the embodiments of the second aspect of the invention.

In an embodiment, the sample is selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries. That is, the present invention provides a method of enriching one or more species of nucleic acid molecules to which the unique 3′ sequences comprised by the nucleotide sequences of the multiple oligonucleotide species covalently attached at their 5′ ends to the particle of any one of the embodiments of the second aspect of the invention are at least 80%, e.g. at least 80, 81, 82, 83, 84, 85, 86, 87, 88, 89, 90, 91, 92, 93, 94, 95, 96, 97, 98, 99, or 99.5%, or 100%, complementary in a sample selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries, wherein the method comprises hybridization-based capture of the one or more species of nucleic acid molecules with the particle of any one of the embodiments of the second aspect of the invention.

In an embodiment, the nucleic acid molecules are RNA molecules or DNA molecules. That is, the present invention provides a method of enriching one or more species of RNA molecules to which the unique 3′ sequences comprised by the nucleotide sequences of the multiple oligonucleotide species covalently attached at their 5′ ends to the particle of any one of the embodiments of the second aspect of the invention are at least 80%, e.g. at least 80, 81, 82, 83, 84, 85, 86, 87, 88, 89, 90, 91, 92, 93, 94, 95, 96, 97, 98, 99, or 99.5%, or 100%, complementary in a sample selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries, wherein the method comprises hybridization-based capture of the one or more species of RNA molecules with the particle of any one of the embodiments of the second aspect of the invention. Further, the present invention provides a method of enriching one or more species of DNA molecules to which the unique 3′ sequences comprised by the nucleotide sequences of the multiple oligonucleotide species covalently attached at their 5′ ends to the particle of any one of the embodiments of the second aspect of the invention are at least 80% complementary in a sample selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries, wherein the method comprises hybridization-based capture of the one or more species of DNA molecules with the particle of any one of the embodiments of the second aspect of the invention.

In a preferred embodiment, the sample is selected from the group consisting of crude tissue lysates, cleared tissue lysates, crude cell lysates, and cleared cell lysates, the sample has been cross-linked, and the nucleic acid molecules have been cross-linked to one or more proteins and/or one or more other nucleic acid molecules. That is, the present invention provides a method of enriching one or more species of nucleic acid molecules to which the unique 3′ sequences comprised by the nucleotide sequences of the multiple oligonucleotide species covalently attached at their 5′ ends to the particle of any one of the embodiments of the second aspect of the invention are at least 80%, e.g. at least 80, 81, 82, 83, 84, 85, 86, 87, 88, 89, 90, 91, 92, 93, 94, 95, 96, 97, 98, 99, or 99.5%, or 100%, complementary in a sample selected from the group consisting of crude tissue lysates, cleared tissue lysates, crude cell lysates, and cleared cell lysates, wherein the sample has been cross-linked, and the nucleic acid molecules have been cross-linked to one or more proteins and/or one or more other nucleic acid molecules, wherein the method comprises hybridization-based capture of the one or more species of nucleic acid molecules with the particle of any one of the embodiments of the second aspect of the invention.

In an embodiment, the nucleic acid molecules are RNA molecules or DNA molecules. That is, the present invention provides a method of enriching one or more species of RNA molecules to which the unique 3′ sequences comprised by the nucleotide sequences of the multiple oligonucleotide species covalently attached at their 5′ ends to the particle of any one of the embodiments of the second aspect of the invention are at least 80%, e.g. at least 80, 81, 82, 83, 84, 85, 86, 87, 88, 89, 90, 91, 92, 93, 94, 95, 96, 97, 98, 99, or 99.5%, or 100%, complementary in a sample selected from the group consisting of crude tissue lysates, cleared tissue lysates, crude cell lysates, and cleared cell lysates, wherein the sample has been cross-linked, and the nucleic acid molecules have been cross-linked to one or more proteins and/or one or more other nucleic acid molecules, wherein the method comprises hybridization-based capture of the one or more species of RNA molecules with the particle of any one of the embodiments of the second aspect of the invention. Further, the present invention provides a method of enriching one or more species of DNA molecules to which the unique 3′ sequences comprised by the nucleotide sequences of the multiple oligonucleotide species covalently attached at their 5′ ends to the particle of any one of the embodiments of the second aspect of the invention are at least 80%, e.g. at least 80, 81, 82, 83, 84, 85, 86, 87, 88, 89, 90, 91, 92, 93, 94, 95, 96, 97, 98, 99, or 99.5%, or 100%, complementary in a sample selected from the group consisting of crude tissue lysates, cleared tissue lysates, crude cell lysates, and cleared cell lysates, wherein the sample has been cross-linked, and the nucleic acid molecules have been cross-linked to one or more proteins and/or one or more other nucleic acid molecules, wherein the method comprises hybridization-based capture of the one or more species of DNA molecules with the particle of any one of the embodiments of the second aspect of the invention.

In an embodiment, in any one of the inventive enrichment methods, each of the unique 3′ sequences comprised by the nucleotide sequences of the multiple DNA oligonucleotide species is complementary to a different stretch of the same species of nucleic acid molecules. In an embodiment, different inventive particles targeting different species of nucleic acid molecules, may be combined in the enrichment methods, thereby enriching a mixture of different targeted nucleic acid molecules.

In a different embodiment, in any one of the inventive enrichment methods, each of the unique 3′ sequences comprised by the nucleotide sequences of the multiple DNA oligonucleotide species is complementary to a different species of nucleic acid molecule.

In any one of the inventive enrichment methods, the 5′ unique sequence of the free DNA oligonucleotide species should only be found in the nucleic acid molecule to be enriched in a sample, but not in other nucleic acid molecules present in the sample.

### Fourth Aspect: Method of Depleting One or More Species of Nucleic Acid Molecules in a Sample

In a fourth aspect, the present invention provides a method of depleting one or more species of nucleic acid molecules to which the unique 3′ sequences comprised by the nucleotide sequences of the multiple oligonucleotide species covalently attached at their 5′ ends to the particle of any one of the embodiments of the second aspect of the invention are at least 80% complementary from a sample, wherein the method comprises hybridization-based capture of the one or more species of nucleic acid molecules with the particle of any one of the embodiments of the second aspect of the invention.

In an embodiment, the nucleic acid molecules are RNA molecules or DNA molecules. That is, the present invention provides a method of depleting one or more species of RNA molecules to which the unique 3′ sequences comprised by the nucleotide sequences of the multiple oligonucleotide species covalently attached at their 5′ ends to the particle of any one of the embodiments of the second aspect of the invention are at least 80% complementary from a sample, wherein the method comprises hybridization-based capture of the one or more species of RNA molecules with the particle of any one of the embodiments of the second aspect of the invention.

Further, the present invention provides a method of depleting one or more species of DNA molecules to which the unique 3′ sequences comprised by the nucleotide sequences of the multiple oligonucleotide species covalently attached at their 5′ ends to the particle of any one of the embodiments of the second aspect of the invention are at least 80% complementary from a sample, wherein the method comprises hybridization-based capture of the one or more species of DNA molecules with the particle of any one of the embodiments of the second aspect of the invention.

In an embodiment, the sample is selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries. That is, the present invention provides a method of depleting one or more species of nucleic acid molecules to which the unique 3′ sequences comprised by the nucleotide sequences of the multiple oligonucleotide species covalently attached at their 5′ ends to the particle of any one of the embodiments of the second aspect of the invention are at least 80% complementary from a sample selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries, wherein the method comprises hybridization-based capture of the one or more species of nucleic acid molecules with the particle of any one of the embodiments of the second aspect of the invention.

In an embodiment, the nucleic acid molecules are RNA molecules or DNA molecules. That is, the present invention provides a method of depleting one or more species of RNA molecules to which the unique 3′ sequences comprised by the nucleotide sequences of the multiple oligonucleotide species covalently attached at their 5′ ends to the particle of any one of the embodiments of the second aspect of the invention are at least 80% complementary from a sample selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries, wherein the method comprises hybridization-based capture of the one or more species of RNA molecules with the particle of any one of the embodiments of the second aspect of the invention. Further, the present invention provides a method of depleting one or more species of DNA molecules to which the unique 3′ sequences comprised by the nucleotide sequences of the multiple oligonucleotide species covalently attached at their 5′ ends to the particle of any one of the embodiments of the second aspect of the invention are at least 80% complementary from a sample selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries, wherein the method comprises hybridization-based capture of the one or more species of DNA molecules with the particle of any one of the embodiments of the second aspect of the invention.

In a preferred embodiment, the sample is selected from the group consisting of crude tissue lysates, cleared tissue lysates, crude cell lysates, and cleared cell lysates, the sample has been cross-linked, and the nucleic acid molecules have been cross-linked to one or more proteins and/or one or more other nucleic acid molecules. That is, the present invention provides a method of depleting one or more species of nucleic acid molecules to which the unique 3′ sequences comprised by the nucleotide sequences of the multiple oligonucleotide species covalently attached at their 5′ ends to the particle of any one of the embodiments of the second aspect of the invention are at least 80% complementary from a sample selected from the group consisting of crude tissue lysates, cleared tissue lysates, crude cell lysates, and cleared cell lysates, wherein the sample has been cross-linked, and the nucleic acid molecules have been cross-linked to one or more proteins and/or one or more other nucleic acid molecules, wherein the method comprises hybridization-based capture of the one or more species of nucleic acid molecules with the particle of any one of the embodiments of the second aspect of the invention.

In an embodiment, the nucleic acid molecules are RNA molecules or DNA molecules. That is, the present invention provides a method of depleting one or more species of RNA molecules to which the unique 3′ sequences comprised by the nucleotide sequences of the multiple oligonucleotide species covalently attached at their 5′ ends to the particle of any one of the embodiments of the second aspect of the invention are at least 80% complementary from a sample selected from the group consisting of crude tissue lysates, cleared tissue lysates, crude cell lysates, and cleared cell lysates, wherein the sample has been cross-linked, and the nucleic acid molecules have been cross-linked to one or more proteins and/or one or more other nucleic acid molecules, wherein the method comprises hybridization-based capture of the one or more species of RNA molecules with the particle of any one of the embodiments of the second aspect of the invention. Further, the present invention provides a method of depleting one or more species of DNA molecules to which the unique 3′ sequences comprised by the nucleotide sequences of the multiple oligonucleotide species covalently attached at their 5′ ends to the particle of any one of the embodiments of the second aspect of the invention are at least 80% complementary from a sample selected from the group consisting of crude tissue lysates, cleared tissue lysates, crude cell lysates, and cleared cell lysates, wherein the sample has been cross-linked, and the nucleic acid molecules have been cross-linked to one or more proteins and/or one or more other nucleic acid molecules, wherein the method comprises hybridization-based capture of the one or more species of DNA molecules with the particle of any one of the embodiments of the second aspect of the invention.

In an embodiment, in any one of the inventive depletion methods, each of the unique 3′ sequences comprised by the nucleotide sequences of the multiple DNA oligonucleotide species is complementary to a different stretch of the same species of nucleic acid molecules. In an embodiment, different inventive particles targeting different species of nucleic acid molecules, may be combined in the depletion methods, thereby depleting a mixture of different targeted nucleic acid molecules.

In a different embodiment, in any one of the inventive depletion methods, each of the unique 3′ sequences comprised by the nucleotide sequences of the multiple DNA oligonucleotide species is complementary to a different species of nucleic acid molecule.

In any one of the inventive depletion methods, the 5′ unique sequence of the free DNA oligonucleotide species should only be found in the nucleic acid molecule to be depleted from a sample, but not in other nucleic acid molecules present in the sample.

### EXAMPLES

The following Examples are merely illustrative and shall describe the present invention in a further way. These Examples shall not be construed to limit the present invention thereto.

### Example 1: Production of a Paramagnetic Particle on the Surface of which Multiple Copies of Each of Multiple DNA Oligonucleotide Species are Covalently Attached at their 5′ Ends

Paramagnetic particles bearing a covalently attached (at its 5′ end) initial oligonucleotide (SEQ ID NO: 1: TTTCCGCACGCTACC) were produced according to the methodology previously described in U.S. Pat. No. 5,512,439 A1. The particles were stored in a storage buffer containing 0.05% Tween-20, 0.02% NaN3, 1×PBS (pH 7.4 @ 25° C.) at 4° C. at a concentration of 5 mg particles per ml of buffer.

### Preparation of the Paramagnetic Particles for the Synthesis Reaction:

The container with paramagnetic particles carrying the initial oligonucleotide covalently attached to their surface was taken from the fridge and allowed to equilibrate to room temperature. Then the container was vortexed until the particles were evenly resuspended in the storage buffer.

The desired amount of paramagnetic particles was transferred to a reaction tube by pipetting. Particles in the tube were concentrated using a magnetic rack and the storage buffer was discarded. Particles were washed twice by careful resuspension in the same volume of washing buffer as the original volume of storage buffer in which the desired amount of paramagnetic particles had been stored. The washing buffer contained 50 nM NaCl, 10 nM Tris pH 7.5 and 0.1% (v/v) Tween-20. Particles were concentrated on a magnetic rack and the washing buffer was discarded.

### Preparation of the Synthesis Reaction Mix:

The reaction mixture containing the following components were assembled and briefly kept on ice until used for the synthesis with magnetic particles:
200 μM dNTPs (dNTP Mix (10 mM each), Thermo Fisher Scientific R0192)
20 units of DNA polymerase per ml (Phusion High-Fidelity DNA Polymerase, Thermo Fisher Scientific F530L)
3 μM free oligonucleotide species with unique 5′ sequences complementary to either human GPDH gene transcript, which has the sequence of SEQ ID NO: 33, (even mixture of free oligonucleotides of SEQ ID NOs: 2-9) or human MALAT1 gene transcript, which has the sequence of SEQ ID NO: 34, (even mixture of free oligonucleotides of SEQ ID NOs: 10-32)
1× concentration of suitable polymerase buffer (Phusion HF buffer, Thermo Fisher Scientific F530L).

TABLE 1
Free oligonucleotide species sequences

| Target | Oligo- | SEQ | |
|---|---|---|---|
| transcript | nucleotide | ID | |
| name | name | NO: | Oligonucleotide sequence (5′ to 3′) |
| GAPDH | GAPDH1 | 2 | GCTCTCTGCTCCTCCTGTTCGACAGGTAGCGTGCGGAAA |
| GAPDH | GAPDH2 | 3 | GCCATCAATGACCCCTTCATTGACCTGGTAGCGTGCGGAAA |
| GAPDH | GAPDH3 | 4 | GCTGGCGCTGAGTACGTCGTGGTAGCGTGCGGAAA |
| GAPDH | GAPDH4 | 5 | CCAACTGCTTAGCACCCCTGGCGGTAGCGTGCGGAAA |
| GAPDH | GAPDH5 | 6 | CCAAGGCTGTGGGCAAGGTCAGGTAGCGTGCGGAAA |
| GAPDH | GAPDH6 | 7 | CCTCAAGGGCATCCTGGGCTACAGGTAGCGTGCGGAAA |
| GAPDH | GAPDH7 | 8 | GGGTGGTGGACCTCATGGCCGGTAGCGTGCGGAAA |
| GAPDH | GAPDH8 | 9 | GAGCCGCACCTTGTCATGTACCATCGGTAGCGTGCGGAAA |
| MALAT1 | MALAT1 | 10 | GGCGCCGGGAAGCCTCAGCTCGGGTAGCGTGCGGAAA |
| MALAT1 | MALAT2 | 11 | GGCCACTTGAACTCGCTTTCCATGGCGATTTGCGGTAGCGTGCGGAAA |
| MALAT1 | MALAT3 | 12 | GTTGGGGGAGAAAGTCCGCCATTTTGCCACTGGTAGCGTGCGGAAA |
| MALAT1 | MALAT4 | 13 | GCCTCCCTCACAAAGGCGGCGGAAGGGGTAGCGTGCGGAAA |
| MALAT1 | MALAT5 | 14 | GGCTCCTGGAGACACGACATAACCAGGAGGGTGGTAGCGTGCGGAAA |
| MALAT1 | MALAT6 | 15 | GGCAGCCAGCGCAGGGGCTTCTGGTAGCGTGCGGAAA |
| MALAT1 | MALAT7 | 16 | GGACTGAGGAGCAAGCGAGCAAGCAGCAGGTAGCGTGCGGAAA |
| MALAT1 | MALAT8 | 17 | GGTAGCAGGCGGCTTGGCTTGGCAGGTAGCGTGCGGAAA |
| MALAT1 | MALAT9 | 18 | GCGAGTGGTTGGTAAAAATCCGTGAGGTCGGCAGGTAGCGTGCGGAAA |
| MALAT1 | MALAT10 | 19 | GGGATGGTCTTAACAGGGAAGAGAGAGGGTGGGGGGTAGCGTGCGGAAA |
| MALAT1 | MALAT11 | 20 | GGCAATTAGTTGGCAGTGGCCTGTTACGGTTGGGGGTAGCGTGCGGAAA |
| MALAT1 | MALAT12 | 21 | GGGGTTGGTCTGGCCTACTGGGCTGACGGTAGCGTGCGGAAA |
| MALAT1 | MALAT13 | 22 | GAGGGTGGGCTTTTGTTGATGAGGGAGGGGAGGTAGCGTGCGGAAA |
| MALAT1 | MALAT14 | 23 | GGGATCAAGTGGATTGAGGAGGCTGTGCTGTGTGGTAGCGTGCGGAAA |
| MALAT1 | MALAT15 | 24 | CCTGACCCCTTCCCTAGGGGATTTCAGGATTGAGAGGTAGCGTGCGGAAA |
| MALAT1 | MALAT16 | 25 | GGGAAGGGAGGGGGTGCCTGTGGGGGTAGCGTGCGGAAA |
| MALAT1 | MALAT17 | 26 | TCTGTAGTTCAGTGTTGGGGCAATCTTGGGGGGGGTAGCGTGCGGAAA |
| MALAT1 | MALAT18 | 27 | TCCTGGAATTTGGAGGGATGGGAGGAGGGGGGTAGCGTGCGGAAA |
| MALAT1 | MALAT19 | 28 | GCAGACACACGTATGCGAAGGGCCAGAGAAGCGGTAGCGTGCGGAAA |
| MALAT1 | MALAT20 | 29 | GGAGGGGTGAGGTGGGCGCTAAGCCGGTAGCGTGCGGAAA |
| MALAT1 | MALAT21 | 30 | GCGGTGCTTGAAGGGGAGGGAAAGGGGGGTAGCGTGCGGAAA |
| MALAT1 | MALAT22 | 31 | GAGTGGCTGAGAGGGCTTTTGGGTGGGAATGCGGTAGCGTGCGGAAA |
| MALAT1 | MALAT23 | 32 | TGGAGTTTTGGGGAGGTGGGAGGTAACAGCACAGGTAGCGTGCGGAAA |

### Synthesis of Hybridization Oligonucleotides on the Surface of the Particles:

5 Paramagnetic magnetic particles were carefully resuspended in 400 μl of the synthesis reaction mix per mg of paramagnetic particles
reactions were preincubated at 94° C. for 1 min
reactions were transferred to the temperature suitable for the annealing of oligonucleotides, thereby forming a DNA duplex (first temperature, in this case 45° C.) for 2 min
reactions were subsequently transferred to 72° C. (second temperature) and incubated for 2 min to allow the process of polymerase elongation
reactions were incubated in 94° C. (third temperature) for 2 min to denature the polymerase-DNA duplex complexes
paramagnetic particles were quickly concentrated on a magnetic rack and the supernatant was discarded
paramagnetic particles were resuspended in the same volume that the desired amount had originally been stored in of washing buffer (50 nM NaCl, 10 nM Tris pH 7.5 and 0.1% (v/v) Tween-20) and incubated again at 94° C. for 2 min, concentrated again, and washing buffer was discarded
washed particles were resuspended in storage buffer (0.05% Tween-20, 0.02% NaN3, 1×PBS (pH 7.4 @ 25° C.)) to achieve the concentration of 5 mg of particles per ml and kept in 4° C. until further use.

### Testing for Successful Probe Synthesis on the Surface of the Paramagnetic Particle:

250 μg (50 μl) of the resulting particle suspensions (5 mg/ml) from
particles carrying multiple DNA oligonucleotide species with unique 3′ sequences complementary to GAPDH
particles carrying multiple DNA oligonucleotide species with unique 3′ sequences complementary to MALAT1
paramagnetic particles bearing the initial oligonucleotide covalently attached at its 5′ end to their surface and not subjected to the production process (negative control)
   was transferred to fresh Eppendorf tubes each, concentrated on a magnetic rack and the buffer was discarded.

Particles were resuspended in 500 μl of Washing Buffer (same as as used in the synthesis described above) by pipetting, concentrated on the magnetic rack and the buffer was discarded. Washed particles were resuspended in 100 μl of the Washing Buffer and 300 μM (3 μl of 100 μM/μl) of an appropriate mixture of free DNA oligonucleotide species complementary to the sequences that were synthesized on a given population of particles was added (i.e. the same free oligonucleotide species used for the synthesis reaction). Mixtures were incubated in a thermo block for 15 min at 55° C. with shaking cycles of 30 sec on/30 sec off at 600 RPM. After the incubation, the particles were concentrated on a magnetic rack and the liquid supernatant was discarded. Next, the particles were resuspended in 10 μl of Elution Buffer (10 mM Tris pH 7.5) and incubated in a thermo block at 80° C. for 2 min. Particles were concentrated on a magnetic rack and the eluates (i.e. the supernatants) were transferred to fresh tubes. The concentration of oligonucleotides in the eluate was measured by nanodrop using optical density.

Based on the concentration measured, the binding capacity of each batch of paramagnetic particles to the free oligonucleotide species was estimated (see Table 2). The particles not subjected to the production process used in this assay served as a negative control. Under the hybridization temperature of this assay the initial oligonucleotides present on the surface of those particles should not bind to the free oligonucleotides since the Tm temperature of the initial oligonucleotides is too low to facilitate the binding to the complementary sequences comprised in the free oligonucleotides (Table 2).

TABLE 2

| | | Free oligonucleotides | amount of ng/pM | binding capacity |
|---|---|---|---|---|
| | Free oligonucleotides | bound by 250 μg of | of assayed | of 1 mg of |
| Particles bearing | sequences | particles (ng) | oligonucleotides | particles (pM) |
| Multiple | SEQ ID Nos: 2-9 | 549.1 | 12.5 | 175.3 |
| olignonucleotide | | | | |
| species comprising | | | | |
| unique 3′ sequences | | | | |
| complementary to one | | | | |
| each of SEQ ID Nos: 2- | | | | |
| 9 (SEQ ID NO: 35-42) | | | | |
| Initial oligonucleotide | SEQ ID Nos: 2-9 | 11.6 | 12.5 | 3.7 |
| (SEQ ID NO: 1) | | | | |
| Multiple | SEQ ID Nos: 10-22 | 613.3 | 11.4 | 215.2 |
| olignonucleotide | | | | |
| species comprising | | | | |
| unique 3′ sequences | | | | |
| complementary to one | | | | |
| each of SEQ ID Nos: | | | | |
| 10-22 (SEQ ID NO: 43- | | | | |
| 65) | | | | |
| Initial oligonucleotide | SEQ ID Nos: 10-22 | 10 | 11.4 | 3.5 |
| (SEQ ID NO: 1) | | | | |

### Testing for Successful Synthesis of Individual Probes on the Surface of the Paramagnetic Particle:

Eight portions, 2 mg (400 μl) each of the resulting particle suspension (5 mg/ml) from particles carrying multiple DNA oligonucleotide species with unique 3′ sequences complementary to GAPDH was transferred to fresh Eppendorf tubes, concentrated on a magnetic rack and the buffer was discarded. Particles were resuspended in 1 ml of Washing Buffer (same as as used in the synthesis described above) by pipetting, concentrated on the magnetic rack and the buffer was discarded. Washed particles were resuspended in 800 μl of the Washing Buffer and 2400 μM (24 μl of 100 μM/μl) of an appropriate free DNA oligonucleotide species complementary to one of the sequences that were synthesized on a population of particles was added (i.e. each of the separate same free oligonucleotide species used for the synthesis reaction). Mixtures were incubated in a thermo block for 15 min at 55° C. with shaking cycles of 30 sec on/30 sec off at 600 RPM. After the incubation, the particles were concentrated on a magnetic rack and the liquid supernatant was discarded. Next, the particles were resuspended in 10 μl of Elution Buffer (10 mM Tris pH 7.5) and incubated in a thermo block at 80° C. for 2 min. Particles were concentrated on a magnetic rack and the eluates (i.e. the supernatants) were transferred to fresh tubes. The concentration of oligonucleotides in the eluate was measured by nanodrop using optical density.

Based on the concentration measured, the binding capacity of the paramagnetic particles to each of the free oligonucleotide species was estimated (see Table 3).

TABLE 3

| | | Free oligonucleotides | amount of ng/pM | binding capacity |
|---|---|---|---|---|
| | Free oligonucleotide | bound by 2 mg of | of assayed | of 1 mg of |
| Particles bearing | sequence | particles (ng) | oligonucleotides | particles (pM) |
| Multiple | SEQ ID No: 2 | 587.7 | 12 | 24.5 |
| olignonucleotide | | | | |
| species comprising | | | | |
| unique 3′ sequences | | | | |
| complementary to one | | | | |
| each of SEQ ID Nos: 2- | | | | |
| 9 (SEQ ID NO: 35-42) | | | | |
| Multiple | SEQ ID No: 3 | 589.1 | 12.6 | 23.4 |
| olignonucleotide | | | | |
| species comprising | | | | |
| unique 3′ sequences | | | | |
| complementary to one | | | | |
| each of SEQ ID Nos: 2- | | | | |
| 9 (SEQ ID NO: 35-42) | | | | |
| Multiple | SEQ ID No: 4 | 532.2 | 11 | 24.2 |
| olignonucleotide | | | | |
| species comprising | | | | |
| unique 3′ sequences | | | | |
| complementary to one | | | | |
| each of SEQ ID Nos: 2- | | | | |
| 9 (SEQ ID NO: 35-42) | | | | |
| Multiple | SEQ ID No: 5 | 546.8 | 11.4 | 24.0 |
| olignonucleotide | | | | |
| species comprising | | | | |
| unique 3′ sequences | | | | |
| complementary to one | | | | |
| each of SEQ ID Nos: 2- | | | | |
| 9 (SEQ ID NO: 35-42) | | | | |
| Multiple | SEQ ID No: 6 | 531.5 | 11.3 | 23.5 |
| olignonucleotide | | | | |
| species comprising | | | | |
| unique 3′ sequences | | | | |
| complementary to one | | | | |
| each of SEQ ID Nos: 2- | | | | |
| 9 (SEQ ID NO: 35-42) | | | | |
| Multiple | SEQ ID No: 7 | 557.9 | 11.8 | 23.6 |
| olignonucleotide | | | | |
| species comprising | | | | |
| unique 3′ sequences | | | | |
| complementary to one | | | | |
| each of SEQ ID Nos: 2- | | | | |
| 9 (SEQ ID NO: 35-42) | | | | |
| Multiple | SEQ ID No: 8 | 531.1 | 11 | 24.1 |
| olignonucleotide | | | | |
| species comprising | | | | |
| unique 3′ sequences | | | | |
| complementary to one | | | | |
| each of SEQ ID Nos: 2- | | | | |
| 9 (SEQ ID NO: 35-42) | | | | |
| Multiple | SEQ ID No: 9 | 596.2 | 12.4 | 24.0 |
| olignonucleotide | | | | |
| species comprising | | | | |
| unique 3′ sequences | | | | |
| complementary to one | | | | |
| each of SEQ ID Nos: 2- | | | | |
| 9 (SEQ ID NO: 35-42) | | | | |

### Example 2: Assaying the Initial Oligonucleotide for Unwanted Binding to Complementary Sequences in a Sample

This assay served to determine if the initial oligonucleotide (SEQ ID NO: 1) (which is present as the 5′ sequence in all of the DNA oligonicleotide species covalently attached at their 5′ ends to the surface) is capable of capturing its complementary sequence under the hybridization conditions intended for the capture of nucleic acid molecules of interest. This capture is not desired and ideally should not be observed in the assay. Particles bearing only the initial oligonucleotide on their surface (SEQ ID NO: 1) were used to assay this. Particles bearing multiple oligonucleotide species comprising unique 3′ sequences complementary to either a human GAPDH DNA (8 different DNA oligonucleotide species covalently attached at their 5′ ends to the particle, SEQ ID NOs: 35-42) or a human MALAT1 DNA (23 different DNA oligonucleotide species covalently attached at their 5′ ends to the particle; SEQ ID NOs: 43-65) generated in Example 1 were also used as positive controls for capture of the nucleic acid.

TABLE 4

| Target | Oligo- | SEQ | |
|---|---|---|---|
| transcript | nucleotide | ID | |
| name | name | NO: | Oligonucleotide sequence (5′ to 3′) |
| GAPDH | GAPDH1p | 35 | TTTCCGCACGCTACCTGTCGAACAGGAGGAGCAGAGAGC |
| GAPDH | GAPDH2p | 36 | TTTCCGCACGCTACCAGGTCAATGAAGGGGTCATTGATGGC |
| GAPDH | GAPDH3p | 37 | TTTCCGCACGCTACCACGACGTACTCAGCGCCAGC |
| GAPDH | GAPDH4p | 38 | TTTCCGCACGCTACCGCCAGGGGTGCTAAGCAGTTGG |
| GAPDH | GAPDH5p | 39 | TTTCCGCACGCTACCTGACCTTGCCCACAGCCTTGG |
| GAPDH | GAPDH6p | 40 | TTTCCGCACGCTACCTGTAGCCCAGGATGCCCTTGAGG |
| GAPDH | GAPDH7p | 41 | TTTCCGCACGCTACCGGCCATGAGGTCCACCACCC |
| GAPDH | GAPDH8p | 42 | TTTCCGCACGCTACCGATGGTACATGACAAGGTGCGGCTC |
| MALAT1 | MALAT1p | 43 | TTTCCGCACGCTACCCGAGCTGAGGCTTCCCGGCGCC |
| MALAT1 | MALAT2p | 44 | TTTCCGCACGCTACCGCAAATCGCCATGGAAAGCGAGTTCAAGTGGCC |
| MALAT1 | MALAT3p | 45 | TTTCCGCACGCTACCAGTGGCAAAATGGCGGACTTTCTCCCCCAAC |
| MALAT1 | MALAT4p | 46 | TTTCCGCACGCTACCCCTTCCGCCGCCTTTGTGAGGGAGGC |
| MALAT1 | MALAT5p | 47 | TTTCCGCACGCTACCACCCTCCTGGTTATGTCGTGTCTCCAGGAGCC |
| MALAT1 | MALAT6p | 48 | TTTCCGCACGCTACCAGAAGCCCCTGCGCTGGCTGCC |
| MALAT1 | MALAT7p | 49 | TTTCCGCACGCTACCTGCTGCTTGCTCGCTTGCTCCTCAGTCC |
| MALAT1 | MALAT8p | 50 | TTTCCGCACGCTACCTGCCAAGCCAAGCCGCCTGCTACC |
| MALAT1 | MALAT9p | 51 | TTTCCGCACGCTACCTGCCGACCTCACGGATTTTTACCAACCACTCGC |
| MALAT1 | MALAT10p | 52 | TTTCCGCACGCTACCCCCCACCCTCTCTCTTCCCTGTTAAGACCATCCC |
| MALAT1 | MALAT11p | 53 | TTTCCGCACGCTACCCCCAACCGTAACAGGCCACTGCCAACTAATTGCC |
| MALAT1 | MALAT12p | 54 | TTTCCGCACGCTACCGTCAGCCCAGTAGGCCAGACCAACCCC |
| MALAT1 | MALAT13p | 55 | TTTCCGCACGCTACCTCCCCTCCCTCATCAACAAAAGCCCACCCTC |
| MALAT1 | MALAT14p | 56 | TTTCCGCACGCTACCACACAGCACAGCCTCCTCAATCCACTTGATCCC |
| MALAT1 | MALAT15p | 57 | TTTCCGCACGCTACCTCTCAATCCTGAAATCCCCTAGGGAAGGGGTCAGG |
| MALAT1 | MALAT16p | 58 | TTTCCGCACGCTACCCCCACAGGCACCCCCTCCCTTCCC |
| MALAT1 | MALAT17p | 59 | TTTCCGCACGCTACCCCCCCCAAGATTGCCCCAACACTGAACTACAGA |
| MALAT1 | MALAT18p | 60 | TTTCCGCACGCTACCCCCCTCCTCCCATCCCTCCAAATTCCAGGA |
| MALAT1 | MALAT19p | 61 | TTTCCGCACGCTACCGCTTCTCTGGCCCTTCGCATACGTGTGTCTGC |
| MALAT1 | MALAT20p | 62 | TTTCCGCACGCTACCGGCTTAGCGCCCACCTCACCCCTCC |
| MALAT1 | MALAT21p | 63 | TTTCCGCACGCTACCCCCCTTTCCCTCCCCTTCAAGCACCGC |
| MALAT1 | MALAT22p | 64 | TTTCCGCACGCTACCGCATTCCCACCCAAAAGCCCTCTCAGCCACTC |
| MALAT1 | MALAT23p | 65 | TTTCCGCACGCTACCTGTGCTGTTACCTCCCACCTCCCCAAAACTCCA |

The oligonucleotide pools originally used for the synthesis of GAPDH and MALAT1 capture particles were used with the particles in the following combinations:
Initial oligonucleotide bearing particles with free oligonucleotides of SEQ ID NOs: 2-9
GAPDH capture particles bearing oligonucleotide species with sequences of SEQ ID NOs: 35-42 with free oligonucleotides of SEQ ID NOs: 2-9
Initial oligonucleotide bearing particles with free oligonucleotides of SEQ ID NOs: 10-32
MALAT1 capture particles bearing oligonucleotide species with sequences of SEQ ID NOs: 43-65 with free oligonucleotides of SEQ ID NOs: 10-32

All free oligonucleotides used comprise a 3′ sequence that is fully complementary to the sequence of the initial oligonucleotide. Therefore, the assay should determine if the experimental conditions are restrictive enough to prevent binding of the initial oligonucleotide to complementary oligonucleotides.

### Preparation of Paramagnetic Particles:

Paramagnetic particles were prepared as described in Example 1.

### Preparation of Hybridization Mixtures:

200 ul of Hybridization/Wash Buffer (100 mM Tris-HCl, pH 7.5; 1% (v/v) Lithium Dodecyl Sulfate; 500 mM Lithium Chloride; 5 mM EDTA; 5 mM Dithiothreitol (DTT)) and 4 μl of specific free oligonucleotide pools at a concentration of 1253 and 1140 ng/tat for GAPDH and MALAT1 oligonucleotides, respectively, was added to the washed particles.

### Hybridization Reaction:

Tubes containing the particle-oligonucleotides mixtures were placed in a thermo block and were incubated for 1 h at 55° C. with shaking cycles of 30 sec on/30 sec off at 900 RPM. After the incubation, particles were concentrated on a magnetic stand and liquid supernatant was discarded. 200 μl of Washing Buffer was added to the sample and particles were washed by incubating the tubes in the thermo block for 5 min at 22° C. with shaking cycles of 30 sec on/30 sec off at 900 RPM. The particles were concentrated on a magnetic rack and the buffer was discarded. The particles were resuspended in 10 μl of elution buffer (10 mM Tris pH 7.5). Elution was performed by incubation in a thermo block at 80° C. for 2 min. After the incubation, particles were quickly concentrated on a magnetic rack and the Buffer containing eluted oligonucleotides was transferred to a fresh tube and used directly to measure the concentration of the single stranded eluted oligonucleotides by nanodrop using optical density.

TABLE 5

| | | concentration |
|---|---|---|
| | | of eluted |
| | Free oligonucleotides | oligonucleotides |
| Particles bearing | sequences | (ng/μl) |
| Initial oligonucleotide | SEQ ID Nos: 2-9 | 3.1 |
| (SEQ ID NO: 1) | | |
| Multiple | SEQ ID Nos: 2-9 | 100.87 |
| olignonucleotide | | |
| species comprising | | |
| unique 3′ sequences | | |
| complementary to one | | |
| each of SEQ ID Nos: 2- | | |
| 9 (SEQ ID NO: 34-42) | | |
| Initial oligonucleotide | SEQ ID Nos: 10-22 | 3.55 |
| (SEQ ID NO: 1) | | |
| Multiple | SEQ ID Nos: 10-22 | 119.01 |
| olignonucleotide | | |
| species comprising | | |
| unique 3′ sequences | | |
| complementary to one | | |
| each of SEQ ID Nos: | | |
| 10-22 (SEQ ID NO: 43- | | |
| 65) | | |

The results of the assay demonstrate that full complementary of the initial oligonucleotide to other nucleic acid molecules is not sufficient to provide an efficient capture of oligonucleotides under conditions intended for capture. At the same time they show that the complementarity of the unique 3′ sequences of the oligonucleotide species pools present on a particles designed to target GAPDH or MALAT1 transcripts to the free oligonucleotide species pools in question is sufficient to allow for their efficient capture under the conditions intended for capture.

### Example 3: Enrichment of Nucleic Acids from Different Samples

The following buffers were used for all enrichment procedures:

### Hybridization/Wash Buffer

| | | |
|---|---|---|
| 100 | mM | Tris-HCl, pH 7.5 |
| 1% | (v/v) | Lithium Dodecyl Sulfate |
| 500 | mM | Lithium Chloride |
| 5 | mM | EDTA |
| 5 | mM | Dithiothreitol (DTT) |

### Washing Buffer

| | | |
|---|---|---|
| 10 | mM | Tris-HCl, pH 7.5 |
| 50 | mM | NaCl |
| 0.5% | (v/v) | Tween-20 |

### Elution Buffer

| | |
|---|---|
| 10 mM | Tris-HCl, pH 7.5 |

### Lysis Buffer

| | | |
|---|---|---|
| 50 | mM | Tris-HCl, pH 7.5 |
| 150 | mM | KCl |
| 2 | mM | EDTA |
| 0.5% | (v/v) | IGEPAL |
| 0.5 | mM | Dithiothreitol (DTT) |

### 2× Hybridization/Wash Buffer

| | | |
|---|---|---|
| 200 | mM | Tris-HCl, pH 7.5 |
| 2% | (v/v) | Lithium Dodecyl Sulfate |

| | |
|---|---|
| 1M | Lithium Chloride |

| | | |
|---|---|---|
| 10 | mM | EDTA |
| 10 | mM | Dithiothreitol (DTT) |

1. Enrichment of Target RNA from Isolated RNA Sample

Preparation of Paramagnetic Particles: Paramagnetic particles prepared in Example 1 were taken from the fridge and equilibrated to room temperature on the bench and resuspended in the Storage Buffer by pipetting. 100 μl of the particles suspension (containing 5 mg/ml of the particles) was transferred to a fresh Eppendorf tube, concentrated on a magnetic rack and the buffer was discarded. Particles were resuspended in 100 ul of Hybridization/Wash Buffer, concentrated on the magnetic rack and the Buffer was discarded.

### Preparation of Hybridization Mixtures:

In a fresh tube, 400 μl of Hybridization/Wash Buffer and 4 μg (for the enrichment of GAPDH) or 12 μg (for the enrichment of MALAT1) of purified whole cell RNA from HEK293 cells were mixed by pipetting. The Buffer containing the RNA was added to the previously prepared paramagnetic particles and particles were resuspended by pipetting. 400 ng of the same purified RNA from HEK293 pool was mixed in a fresh tube with Elution Buffer to a final volume of 40 μl and saved for later analysis.

### Hybridization Reaction:

Tubes containing the particle-RNA mixture were placed in a thermo block and incubated for 1 h at 55° C. with shaking cycles of 30 sec on/30 sec off at 900 RPM. After incubation, particles in tubes were concentrated on a magnetic stands, and liquid supernatant was discarded. To wash, 400 μl of Hybridization/Wash Buffer was added to the sample and particles were washed by incubating the tubes in the thermo block for 10 min at 55° C. with shaking cycles of 30 sec on/30 sec off at 900 RPM. Next, the particles were concentrated on a magnetic rack and the Buffer was discarded. These washing steps were repeated for a total of 3 washes. After the third wash, particles were washed one more time in 1 ml of Washing Buffer by incubating the tubes in the thermo block for 5 min at 22° C. with shaking cycles of 30 sec on/30 sec off at 900 RPM. Particles were concentrated on a magnetic rack and resuspended by pipetting in 40 ul of Elution Buffer. Elution was performed by incubation in a thermo block at 80° C. for 2 min. After incubation, particles were quickly concentrated on a magnetic rack and the Buffer containing eluted RNA was transferred to a fresh tube and used directly for downstream analysis or stored in −80° C. for later use.

2. Enrichment of Target RNA from Cellular Lysate

### Preparation of Paramagnetic Particles:

Paramagnetic particles prepared in Example 1 were taken from the fridge and equilibrated to room temperature on the bench and resuspended in the Storage Buffer by pipetting. 100 μl of the particles suspension (containing 5 mg/ml of the particles) was transferred to a fresh Eppendorf tube, concentrated on a magnetic rack and the buffer was discarded. Particles were resuspended in 100 ul of Hybridization/Wash Buffer, concentrated on the magnetic rack and the Buffer was discarded.

### Preparation of the Cellular Lysate for Hybridization:

A tube containing 200 μl of HEK293 cellular pellet was taken from −80° C. and placed on ice to thaw. After thawing, 600 μl of Lysis Buffer was added to the cells and mixed by pipetting. The tube was incubated on ice for 10 min and centrifuged for 10 min at 13000 g and 4° C. to pellet the insoluble cellular components. The supernatant was transferred to a fresh tube and mixed with an equal volume of the 2× Hybridization/Wash Buffer. 400 μl of the lysate was added to the previously prepared paramagnetic particles and the particles were resuspended by pipetting. A 100 μl aliquot of the lysate was instead transferred to a fresh tube and subjected to a standard Phenol/Chloroform RNA extraction, with elution with isopropanol, and resuspension of precipitated nucleic acids in 40 μl of Elution Buffer and saved to serve as an input sample in the downstream analysis.

### Hybridization Reaction:

The tube containing the particle-lysate mixture was placed in a thermo block and was incubated for 1 h at 55° C. with shaking cycles of 30 sec on/30 sec off at 900 RPM. After incubation, particles were concentrated on a magnetic stand and liquid supernatant was discarded. To wash, 400 μl of Hybridization/Wash Buffer was added to the sample and particles were washed by incubating the tubes in the thermo block for 10 min at 55° C. with shaking cycles of 30 sec on/30 sec off at 900 RPM. The particles were then concentrated on a magnetic rack and the Buffer was discarded. These washing steps were repeated for the total of 3 washes. After the third wash, particles were washed one more time in 1 ml of Washing Buffer by incubating the tubes in the thermo block for 5 min in 22° C. with shaking cycles of 30 sec on/30 sec off at 900 RPM. Particles were concentrated on a magnetic rack and resuspended by pipetting in 40 μl of Elution Buffer. Elution was performed by incubation in a thermo block at 80° C. for 2 min. After incubation, particles were quickly concentrated on a magnetic rack and the Buffer containing eluted nucleic acids was transferred to a fresh tube and used directly for downstream analysis or stored in −80° C. for later use.

3. Readout of the Enrichment Efficiency Analysis:

All nucleic acid samples from the above described enrichment procedures (input samples and enriched nucleic acid samples eluted from the particles). Were subjected to analysis by RT-qPCR to assay the efficiency of the target nucleic acid molecule capture.

First, cDNA was synthesized from each sample using Super Script III from Thermo Fisher Scientific with 10 μl of each nucleic acid sample and both oligo dT and random primers according to the manufacturers protocol using the components provided in Table 6 below. Resulting cDNA was diluted to a volume of 200 μl and subjected to RT-qPCR reactions with LightCycler 480 SYBR Green I Master from Roche with the total reaction volume of 10 μl using 3 μl of the cDNA and appropriate primers according to the manufacturers protocol. The reactions were run using the LightCycler 96 Instrument from Roche and software provided by the instrument manufacturer:

TABLE 6
cDNA synthesis components

| | | Catalog |
|---|---|---|
| Component name | Supplier | number |
| SuperScript III Reverse | Thermo Fisher Scientific | 18080044 |
| Transcriptase | | |
| 100 mM DTT | Thermo Fisher Scientific | 18080044 |
| 5X first-strand buffer | Thermo Fisher Scientific | 18080044 |
| dNTP Mix (10 mM each) | Thermo Fisher Scientific | R0192 |
| RiboLock RNase Inhibitor | Thermo Fisher Scientific | EO0381 |
| Oligo(dT)18 Primer | Thermo Fisher Scientific | SO132 |
| Random Hexamer Primer | Thermo Fisher Scientific | SO142 |

For every input and enriched sample, RT-qPCR reactions were run in duplicates or triplicates using primers amplifying GAPDH, MALAT1, ACTB and 18S rRNA transcript cDNAs.

TABLE 7
RT-qPCR reaction profile:

| Step | Temperature | Time | |
|---|---|---|---|
| Preincubation | 95° C. | 10 | min |
| Amplification (repeated 45×) | 95° C. | 10 | s |
| | 60° C. | 10 | s |
| | 72° C. | 10 | s |
| Melting Curve | 95° C. | 10 | s |
| | 65° C. | 1 | min |
| | 97° C. | 1 | s |

TABLE 8
Components used for RT-qPCR:

| Component name | Supplier | Catalog number |
|---|---|---|
| LightCycler 480 SYBR Green I Master | Roche | 4887352001 |

TABLE 9
Oligonucleotides used for RT-qPCR reactions

| Target | | | | |
|---|---|---|---|---|
| transcript | Forward | Forward primer sequence | Reverse | Reverse primer sequence |
| name | primer name | (5′ to 3′) | primer name | (5′ to 3′) |
| GAPDH | GAPDHqF | GTCTCCTCTGACTTCAACAGCG | GAPDHqR | ACCACCCTGTTGCTGTAGCCAA |
| | | (SEQ ID NO: 66) | | (SEQ ID NO: 67) |
| MALAT1 | MALAT1qF | GACGGAGGTTGAGATGAAGC | MALAT1qR | ATTCGGGGCTCTGTAGTCCT |
| | | (SEQ ID NO: 68) | | (SEQ ID NO: 69) |
| 18S rRNA | hm18SqF | GTAACCCGTTGAACCCCATT | hm18SqR | CCATCCAATCGGTAGTAGCG |
| | | (SEQ ID NO: 70) | | (SEQ ID NO: 71) |
| ACTB | hACTqF | AGGCACCAGGGCGTGAT (SEQ | hACTqR | GCCCACATAGGAATCCTTCTGAC |
| | | ID NO: 72) | | (SEQ ID NO: 73) |

4. Analysis of RT-qPCR Results:

The data obtained from the RT-qPCR measurements was processed in a standard way for assessing the enrichment efficiency of RNA pull down experiments. The mean Cq values from technical replicates for each transcript amplified in RT-qPCR recorded and calculated by the LightCycler 96 Instrument from Roche software were transformed by the following equation: 2\hat{ }-Cq (see Table 10).

TABLE 10
Results of RT-qPCR experiment run with the LightCycler 96 Instrument from Roche
containing mean Cq values transformed with the 2\hat{ } − Cq formula.

| | | | | | |
|---|---|---|---|---|---|
| | Assayed | | | | |
| Sample Name | transcript | Cq | Cq Mean | Cq Error | 2\hat{ } − Cq Mean |

Enrichment from isolated RNA samples

| | | | | | |
|---|---|---|---|---|---|
| Pull Down GAPDH | GAPDH | 15.7 | 15.84 | 0.121655 | 1.70484E−05 |
| Pull Down GAPDH | GAPDH | 15.92 | 15.84 | 0.121655 | 1.70484E−05 |
| Pull Down GAPDH | GAPDH | 15.9 | 15.84 | 0.121655 | 1.70484E−05 |
| Pull Down GAPDH | ACTB | 29.13 | 29.27 | 0.277849 | 1.54473E−09 |
| Pull Down GAPDH | ACTB | 29.09 | 29.27 | 0.277849 | 1.54473E−09 |
| Pull Down GAPDH | ACTB | 29.59 | 29.27 | 0.277849 | 1.54473E−09 |
| Pull Down GAPDH | MALAT1 | | | | |
| Pull Down GAPDH | MALAT1 | 37.99 | 37.99 | 0 | 3.66328E−12 |
| Pull Down GAPDH | MALAT1 | | | | |
| Pull Down GAPDH | hm18S | 24.96 | 25.05667 | 0.134288 | 2.86544E−08 |
| Pull Down GAPDH | hm18S | 25.21 | 25.05667 | 0.134288 | 2.86544E−08 |
| Pull Down GAPDH | hm18S | 25 | 25.05667 | 0.134288 | 2.86544E−08 |
| Pull Down MALAT1 | GAPDH | 32.08 | 31.31 | 0.667757 | 3.75622E−10 |
| Pull Down MALAT1 | GAPDH | 30.89 | 31.31 | 0.667757 | 3.75622E−10 |
| Pull Down MALAT1 | GAPDH | 30.96 | 31.31 | 0.667757 | 3.75622E−10 |
| Pull Down MALAT1 | ACTB | 31.34 | 31.14667 | 0.292632 | 4.20648E−10 |
| Pull Down MALAT1 | ACTB | 31.29 | 31.14667 | 0.292632 | 4.20648E−10 |
| Pull Down MALAT1 | ACTB | 30.81 | 31.14667 | 0.292632 | 4.20648E−10 |
| Pull Down MALAT1 | MALAT1 | 21.73 | 21.70667 | 0.049329 | 2.92174E−07 |
| Pull Down MALAT1 | MALAT1 | 21.74 | 21.70667 | 0.049329 | 2.92174E−07 |
| Pull Down MALAT1 | MALAT1 | 21.65 | 21.70667 | 0.049329 | 2.92174E−07 |
| Pull Down MALAT1 | hm18S | 21.98 | 21.98667 | 0.005774 | 2.40632E−07 |
| Pull Down MALAT1 | hm18S | 21.99 | 21.98667 | 0.005774 | 2.40632E−07 |
| Pull Down MALAT1 | hm18S | 21.99 | 21.98667 | 0.005774 | 2.40632E−07 |
| Input | GAPDH | 19 | 18.92 | 0.091652 | 2.0161E−06 |
| Input | GAPDH | 18.82 | 18.92 | 0.091652 | 2.0161E−06 |
| Input | GAPDH | 18.94 | 18.92 | 0.091652 | 2.0161E−06 |
| Input | ACTB | 20.39 | 20.55667 | 0.187705 | 6.48376E−07 |
| Input | ACTB | 20.76 | 20.55667 | 0.187705 | 6.48376E−07 |
| Input | ACTB | 20.52 | 20.55667 | 0.187705 | 6.48376E−07 |
| Input | MALAT1 | 26.37 | 26.55 | 0.190788 | 1.01778E−08 |
| Input | MALAT1 | 26.53 | 26.55 | 0.190788 | 1.01778E−08 |
| Input | MALAT1 | 26.75 | 26.55 | 0.190788 | 1.01778E−08 |
| Input | hm18S | 10.08 | 10.25333 | 0.150444 | 0.000819293 |
| Input | hm18S | 10.35 | 10.25333 | 0.150444 | 0.000819293 |
| Input | hm18S | 10.33 | 10.25333 | 0.150444 | 0.000819293 |

Enrichment from cellular lysate

| | | | | | |
|---|---|---|---|---|---|
| Pull down GAPDH | GAPDH | 10.74 | 10.78333 | 0.045092 | 0.000567405 |
| Pull down GAPDH | GAPDH | 10.78 | 10.78333 | 0.045092 | 0.000567405 |
| Pull down GAPDH | GAPDH | 10.83 | 10.78333 | 0.045092 | 0.000567405 |
| Pull down GAPDH | ACTB | 20.59 | 20.61 | 0.02 | 6.24844E−07 |
| Pull down GAPDH | ACTB | 20.63 | 20.61 | 0.02 | 6.24844E−07 |
| Pull down GAPDH | ACTB | 20.61 | 20.61 | 0.02 | 6.24844E−07 |
| Pull down GAPDH | MALAT1 | 26.03 | 26.04333 | 0.032146 | 1.44602E−08 |
| Pull down GAPDH | MALAT1 | 26.08 | 26.04333 | 0.032146 | 1.44602E−08 |
| Pull down GAPDH | MALAT1 | 26.02 | 26.04333 | 0.032146 | 1.44602E−08 |
| Pull down GAPDH | hm18S | 18.48 | 18.51667 | 0.032146 | 2.66642E−06 |
| Pull down GAPDH | hm18S | 18.54 | 18.51667 | 0.032146 | 2.66642E−06 |
| Pull down GAPDH | hm18S | 18.53 | 18.51667 | 0.032146 | 2.66642E−06 |
| Pull down MALAT1 | GAPDH | 28.6 | 28.78 | 0.167033 | 2.16949E−09 |
| Pull down MALAT1 | GAPDH | 28.81 | 28.78 | 0.167033 | 2.16949E−09 |
| Pull down MALAT1 | GAPDH | 28.93 | 28.78 | 0.167033 | 2.16949E−09 |
| Pull down MALAT1 | ACTB | 32.18 | 31.52667 | 0.610765 | 3.23242E−10 |
| Pull down MALAT1 | ACTB | 31.43 | 31.52667 | 0.610765 | 3.23242E−10 |
| Pull down MALAT1 | ACTB | 30.97 | 31.52667 | 0.610765 | 3.23242E−10 |
| Pull down MALAT1 | MALAT1 | 17.73 | 17.58 | 0.130767 | 5.10379E−06 |
| Pull down MALAT1 | MALAT1 | 17.52 | 17.58 | 0.130767 | 5.10379E−06 |
| Pull down MALAT1 | MALAT1 | 17.49 | 17.58 | 0.130767 | 5.10379E−06 |
| Pull down MALAT1 | hm18S | 24.24 | 24.31 | 0.06245 | 4.80796E−08 |
| Pull down MALAT1 | hm18S | 24.36 | 24.31 | 0.06245 | 4.80796E−08 |
| Pull down MALAT1 | hm18S | 24.33 | 24.31 | 0.06245 | 4.80796E−08 |
| Input | GAPDH | 12.72 | 12.75 | 0.026458 | 0.000145167 |
| Input | GAPDH | 12.77 | 12.75 | 0.026458 | 0.000145167 |
| Input | GAPDH | 12.76 | 12.75 | 0.026458 | 0.000145167 |
| Input | ACTB | 17.59 | 17.61667 | 0.025166 | 4.97571E−06 |
| Input | ACTB | 17.64 | 17.61667 | 0.025166 | 4.97571E−06 |
| Input | ACTB | 17.62 | 17.61667 | 0.025166 | 4.97571E−06 |
| Input | MALAT1 | 19 | 18.96333 | 0.035119 | 1.95645E−06 |
| Input | MALAT1 | 18.93 | 18.96333 | 0.035119 | 1.95645E−06 |
| Input | MALAT1 | 18.96 | 18.96333 | 0.035119 | 1.95645E−06 |
| Input | hm18S | 9.85 | 9.643333 | 0.179258 | 0.001250453 |
| Input | hm18S | 9.55 | 9.643333 | 0.179258 | 0.001250453 |
| Input | hm18S | 9.53 | 9.643333 | 0.179258 | 0.001250453 |

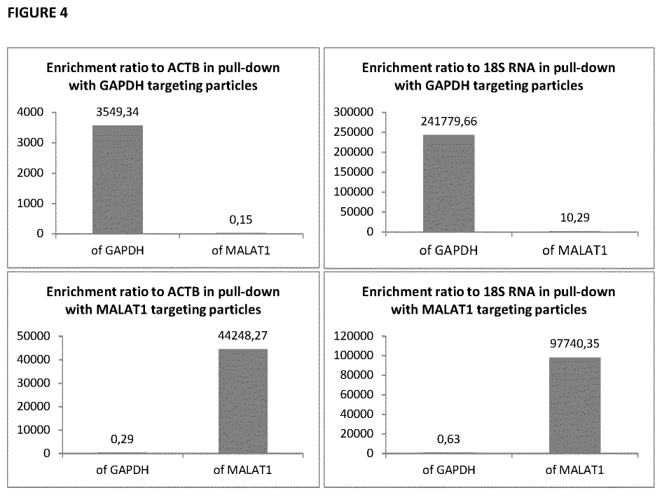

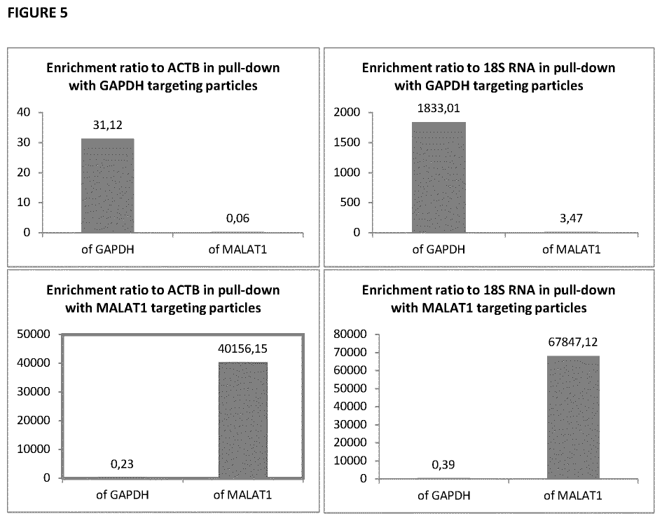

Values obtained for GAPDH and MALAT1 transcripts in every sample were then divided by the values obtained for ACTB and 18S RNA transcripts, providing a ratio of the measured transcripts in each sample. The calculated ratios in the enriched sample were then divided by the corresponding ratios obtained for input samples, resulting in the fold enrichment value over input (see Table 11, FIGS. 4 and 5).

TABLE 11
Calculation of the RT-qPCR results into transcript to transcript
ratios and pull down enrichment values over input.

| | | | | |
|---|---|---|---|---|
| | Ratio Of | Ratio To | | Enrichment Ratio |
| Sample Name | Transcript | Transcript | Ratio Value | Over Input |

Enrichment from isolated RNA

| | | | | |
|---|---|---|---|---|
| Pull down GAPDH | GAPDH | ACTB | 11036.53746 | 3549.33574 |
| Pull down MALAT1 | GAPDH | ACTB | 0.892959511 | 0.287174589 |
| Input | GAPDH | ACTB | 3.109465621 | 1 |
| Pull down GAPDH | MALAT1 | ACTB | 0.002371474 | 0.151074632 |
| Pull down MALAT1 | MALAT1 | ACTB | 694.5814157 | 44248.26704 |
| Input | MALAT1 | ACTB | 0.01569737 | 1 |
| Pull down GAPDH | GAPDH | 18S rRNA | 594.9673405 | 241779.6563 |
| Pull down MALAT1 | GAPDH | 18S rRNA | 0.001560979 | 0.634342247 |
| Input | GAPDH | 18S rRNA | 0.002460783 | 1 |
| Pull down GAPDH | MALAT1 | 18S rRNA | 0.000127844 | 10.29115736 |
| Pull down MALAT1 | MALAT1 | 18S rRNA | 1.214194884 | 97740.35111 |
| Input | MALAT1 | 18S rRNA | 1.24227E−05 | 1 |

Enrichment from cellular lysate

| | | | | |
|---|---|---|---|---|
| Pull down GAPDH | GAPDH | ACTB | 908.0743825 | 31.12495832 |
| Pull down MALAT1 | GAPDH | ACTB | 6.711646198 | 0.230046913 |
| Input | GAPDH | ACTB | 29.17511963 | 1 |
| Pull down GAPDH | MALAT1 | ACTB | 0.023142149 | 0.058856001 |
| Pull down MALAT1 | MALAT1 | ACTB | 15789.37743 | 40156.14992 |
| Input | MALAT1 | ACTB | 0.393199484 | 1 |
| Pull down GAPDH | GAPDH | 18S rRNA | 212.7969014 | 1833.011345 |
| Pull down MALAT1 | GAPDH | 18S rRNA | 0.045122787 | 0.388683203 |
| Input | GAPDH | 18S rRNA | 0.116091426 | 1 |
| Pull down GAPDH | MALAT1 | 18S rRNA | 0.005423099 | 3.466148183 |
| Pull down MALAT1 | MALAT1 | 18S rRNA | 106.1529019 | 67847.12205 |
| Input | MALAT1 | 18S rRNA | 0.00156459 | 1 |

These results demonstrate efficient and specific enrichment of the target transcripts in pull down experiments.

The ratios of the target transcripts (GAPDH or MALAT1) to non-target transcripts (ACTB or 18S RNA) in pull down samples in relation to those ratios in input samples show the fold enrichment of the target over non-target transcript. Successful enrichment of the target nucleic acid molecules is evident in every provided example.

The ratios of non-target transcript MALAT1 (enriched with GAPDH-targeting particles) and GAPDH (enriched with MALAT1-targeting particles) to ACTB and 18S RNA provide further evidence of the specificity of the inventive magnetic particles in target nucleic acid molecule capture. Non-target transcripts were either not enriched or only modestly enriched when compared to the levels of enrichment of the target transcript in every provided example (see FIGS. 4 and 5).

### Example 4: Enrichment of Multiple Nucleic Acid Targets in a Single Reaction

The simultaneous enrichment of multiple different target nucleic acids in one enrichment reaction can be achieved with the same experimental procedures as laid out in Example 3 with minor modifications in two possible ways.

The first is to use the same experimental procedure as in Example 3 with the difference that two or more populations of particles each made to target a specific, distinct nucleic acid molecule are mixed together prior to addition to the sample in which to enrich the target nucleic acid molecules. This can be done in equal or varying ratios.

The second way is to use the same experimental procedure as in Example 3 instead using a single population of particles made to simultaneously target more than one nucleic acid molecule, i.e. in which the unique 3′ sequences of the multiple oligonucleotide covalently linked to the particle are complementary to sequences that are divided between multiple nucleic acid molecules to be enriched. Such particles can be synthesized in the manner described in Example 1 by adding corresponding free oligonucleotide species to the synthesis mixture.

In both instances, the outcome of the enrichment of multiple nucleic acid targets in a single reaction can be assessed in the same way as described in Example 3, except that for each enriched sample, RT-qPCR reactions should be performed separately for all nucleic acid molecules targeted in the enrichment procedure instead of just one.

### Example 5: Enrichment of the Molecules Associated with Target Nucleic Acid Molecules

Enrichment of molecules (e.g. nucleic acids, polypeptides, proteins) that have been cross-linked to a target nucleic acid molecule is performed as described in Example 3, except that a sample previously subjected to chemical or UV light induced cross-linking of proteins to nucleic acids for enrichment purposes. The cross-linking introduces covalent bonds between the nucleic acids of interest and their interacting proteins, allowing for the preservation of the protein-nucleic acid interactions under the conditions of the enrichment procedure. In consequence, enrichment of the nucleic acid molecules of interest will also lead to enrichment of the specific proteins associated with them, which can be subjected to downstream processing and analysis with various methodologies, including mass spectrometry.

### Example 6: Production of a Paramagnetic Particle on the Surface of which Multiple Copies of Each of Multiple DNA Oligonucleotide Species are Covalently Attached at their 5′ Ends Using Free RNA Oligonucleotides and an RNA-Dependent DNA-Polymerase

Reagents and procedures in steps preceding the preparation of the synthesis reaction are identical to Example 1.

### Preparation of the Synthesis Reaction Mix and Synthesis of Hybridization Oligonucleotides on the Surface of the Particles:

A mixture containing the following components is assembled and briefly kept on ice until used for synthesis with magnetic particles:
155 uM dNTPs
4.65 uM template oligonucleotides
Sterile, distilled water

To the washed particles, 260 μl of the mixture was added per each mg of the particles. Then, particles are carefully resuspended in the mixture and incubated at 65° C. for 5 min and subsequently chilled on ice.

Next, the following reaction components are added to the tube containing the particles:
80 μl of 5× concentrated reaction buffer (250 mM Tris-HCl (pH 8.3), 375 mM KCl, 15 mM Magnesium Chloride) per mg of particles
40 μl of 100 mM DTT per mg of particles.

The content of the tubes is mixed gently and incubated at 37° C. for 2 min. After incubation, 20 μl (4000 units) of M-MLV Reverse Transcriptase is added to the tube containing the particles, mixed gently, and incubated first at 25° C. for 10 min (first temperature) for hybridization and subsequently at 37° C. (second temperature) for 50 min for elongation. Next, the reactions are inactivated by incubating at 70° C. for 15 minutes and particles are quickly concentrated on a magnetic rack and the supernatant is discarded. Particles are resuspended in original bead volume of washing buffer (50 nM NaCl, 10 nM Tris pH 7.5 and 0.1% (v/v) Tween-20) and incubated again at 94° C. (third temperature, for denaturing) for 2 min, concentrated again, and washing buffer is discarded. Washed particles are resuspended in storage buffer (0.05% Tween-20, 0.02% NaN3, 1×PBS (pH 7.4 @ 25° C.)) to achieve a concentration of 5 mg of particles per ml and kept in 4° C. until further use.

Testing for successful probe synthesis on the surface of the paramagnetic particle is performed as in Example 1.

1. A method of producing a surface on which multiple copies of each of multiple DNA oligonucleotide species are covalently attached at their 5′ ends, wherein the oligonucleotide species each have a predetermined nucleotide sequence comprising a 3′ sequence that is unique for each oligonucleotide species, the method comprising:
a. contacting a surface on which multiple copies of an initial DNA oligonucleotide are covalently attached at their 5′ ends, wherein the initial DNA oligonucleotide has a predetermined nucleotide sequence, with
i. a DNA-dependent DNA polymerase;
ii. deoxyribonucleotide triphosphates;
iii. a reaction buffer suitable for DNA hybridization and elongation by the DNA-dependent DNA polymerase; and
iv. multiple copies of multiple free DNA oligonucleotide species, wherein the multiple free DNA oligonucleotide species each have a predetermined nucleotide sequence comprising
a 3′ sequence that is complementary to a 3′ sequence of the nucleotide sequence of the initial oligonucleotide that is covalently attached to the surface, and
a 5′ sequence that is unique for each of the multiple free oligonucleotide species;
such that a reaction mixture is formed;
b. incubating the reaction mixture at a first temperature such that the multiple copies of the multiple free DNA oligonucleotide species hybridize to the multiple copies of the initial DNA oligonucleotide that are covalently attached to the surface, wherein the first temperature is a temperature at which a DNA duplex between the sequence of one copy of the covalently attached initial DNA oligonucleotide and the complementary 3′ sequence of one copy of one of the multiple free DNA oligonucleotide species can form with free DNA oligonucleotide species;
c. incubating the reaction mixture at a second temperature such that the multiple copies of the initial DNA oligonucleotide that are covalently attached to the surface elongate, wherein the second temperature is a temperature suitable for binding of the DNA-dependent DNA polymerase to the duplex formed in step b, thereby forming a polymerase-DNA complex, and for attaching the deoxyribonucleotide triphosphates to the 3′ end of the covalently attached initial oligonucleotide using the hybridized oligonucleotide species as a template;
d. incubating the reaction mixture at a third temperature such that the polymerase-DNA complex and duplex formed in step c are denatured, wherein the third temperature is a temperature suitable to form a denatured reaction mixture; and
e. separating the surface from the denatured reaction mixture of step d.

2. The method of claim 1, wherein the surface is the surface of a particle.

3. The method of claim 1, wherein the DNA-dependent DNA polymerase is selected from the group consisting of DNA-dependent DNA polymerase that produces blunt ends and DNA-dependent DNA polymerase that produces sticky ends.

4. The method of claim 1, wherein
the first temperature is from 25° C. to 72° C. and/or
wherein the second temperature is from 40° C. to 78° C., and/or
wherein the third temperature is from 90° C. to 98° C.

5. The method of claim 1, wherein the initial oligonucleotide that is covalently attached to the surface is from 5 nucleotides to 100 nucleotides in length.

6. The method of claim 1, wherein the free oligonucleotide species are from 10 nucleotides to 1000 nucleotides in length.

7. The method of claim 1, wherein the 3′ sequence of the free oligonucleotide species that is complimentary to the 3′ sequence of the covalently attached initial oligonucleotide is from 5 nucleotides to 100 nucleotides in length.

8. A particle on the surface of which multiple copies of each of multiple DNA oligonucleotide species are covalently attached at their 5′ ends, wherein the DNA oligonucleotide species each have a predetermined nucleotide sequence, and wherein the predetermined nucleotide sequence of each DNA oligonucleotide species comprises a 3′ sequence that is unique for each of the DNA oligonucleotide species.

9. The particle of claim 8, wherein the DNA oligonucleotide species are from 10 nucleotides to 1000 nucleotides in length.

10. The particle of claim 8, wherein the unique 3′ sequence is from 5 nucleotides to 995 nucleotides in length.

11. A method of enriching one or more species of nucleic acid molecules to which the unique 3′ sequences comprised by the nucleotide sequences of the multiple oligonucleotide species are at least 80% complementary in a sample, wherein the method comprises hybridization-based capture of the one or more species of nucleic acid molecules with the particle of claim 8.

12. A method of depleting one or more species of nucleic acid molecules to which the unique 3′ sequences comprised by the nucleotide sequences of the multiple oligonucleotide species are at least 80% complementary from a sample, wherein the method comprises hybridization-based capture of the one or more species of nucleic acid molecules with the particle of claim 8.

13. The method of claim 11, wherein the nucleic acid molecules are RNA molecules or DNA molecules.

14. The method of claim 11, wherein the sample is selected from the group consisting of partially isolated nucleic acids, isolated nucleic acids, biological samples, crude tissue lysates, cleared tissue lysates, crude cell lysates, cleared cell lysates, and processed and amplified nucleic acid sequencing libraries.

15. The method of claim 11, wherein each of the unique 3′ sequences comprised by the nucleotide sequences of the multiple oligonucleotide species is complementary to a different stretch of the same species of nucleic acid molecules or wherein each of the unique 3′ sequences comprised by the nucleotide sequences of the multiple oligonucleotide species is complementary to a different species of nucleic acid molecule.

16. The method of claim 2, wherein the particle is a magnetic particle, and the separating in step e is magnetically separating

17. The method of claim 16, wherein the magnetic particle is a paramagnetic particle, and the separating in step e is magnetically separating.

18. The method of claim 3, wherein the DNA-dependent DNA polymerase is a DNA-dependent DNA polymerase that produces blunt ends.

19. The method of claim 4, wherein the second temperature is from 60° C. to 78° C.

20. The method of claim 4, wherein both the first and second temperatures are from 40° C. to 72° C. and steps b and c are performed concurrently.

21. The method of claim 5, wherein the initial oligonucleotide that is covalently attached to the surface is from 10 nucleotides to 20 nucleotides in length.

22. The method of claim 6, wherein the free oligonucleotide species are from 24 nucleotides to 50 nucleotides in length.

23. The method of claim 7, wherein the 3′ sequence of the free oligonucleotide species that is complimentary to the 3′ sequence of the covalently attached initial oligonucleotide is 10 nucleotides to 20 nucleotides in length.

24. The particle of claim 8, wherein the particle is a magnetic particle.

25. The particular of claim 24, wherein the particle a paramagnetic particle.

26. The particle of claim 9, wherein the DNA oligonucleotide species are from 24 nucleotides to 70 nucleotides in length.

27. The particle of claim 10, wherein the unique 3′ sequence is 12 nucleotides to 50 nucleotides in length.

28. The method of claim 14, wherein
the sample is selected from the group consisting of crude tissue lysates, cleared tissue lysates, crude cell lysates, and cleared cell lysates,
wherein the sample has been cross-linked, and
wherein the nucleic acid molecules have been cross-linked to one or more proteins and/or one or more other nucleic acid molecules.
